(19) United States
(12) Patent Application Publication
(10) Pub. No.: US 2007/0269875 A1
(43) Pub. Date: Nov. 22, 2007

(54) Method and apparatus for cell permeabilization

(76) Inventors: Manfred R. Koller, San Diego, CA (US); Elie G. Hanania, Poway, CA (US); Rolf Brandes, San Diego, CA (US); Timothy M. Eisfeld, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/711,426

(22) Filed: Feb. 27, 2007

Related U.S. Application Data
(63) Continuation of application No. 10/698,343, filed on Oct. 31, 2003, now abandoned.

(51) Int. Cl. C12N 13/00; C12M 1/42

(52) U.S. Cl. 435/173.5; 435/283.1; 700/306

(57) ABSTRACT

The invention relates to methods and apparatuses for introducing and releasing substances into and out of cells, and more specifically to methods and apparatuses for transiently permeabilizing a living cell so that any one or more of a variety of substances, such as ions, proteins, and nucleic acids, can be loaded into or released out of the cell.

### CROSS REFERENCE TO RELATED APPLICATIONS

This application claims priority to U.S. application Ser. No. 10/698,343, filed Oct. 31, 2003, which is incorporated herein by reference in its entirety.

### BACKGROUND OF THE INVENTION

1. Field of the Invention

The invention relates to methods and apparatuses for introducing and releasing substances into and out of cells, and more specifically to methods and apparatuses for transiently permeabilizing a living cell so that any one or more of a variety of substances, such as ions, proteins, and nucleic acids, can be loaded into or released out of the cell.

2. Description of the Related Art

The importance of introducing substances into cells and the lack of any ideal procedure has resulted in the development of numerous techniques. For example, DNA is introduced by methods such as calcium phosphate precipitation, liposomes, cationic lipids, DEAE-dextran, viral vectors, electroporation, polyethylenimines, peptide-mediated gene delivery, activated dendrimers, polyamines, poly-L-ornithine and bead based methods such as bolistics, bead-loading and immunoporation. These methods generally suffer from a number of disadvantages, including (i) applicability to only one substance to be introduced, (ii) harmful effects on the cell (e.g., reduced cell viability and growth, altered physiology), (iii) harmful effects on the organism (e.g., induction of leukemia), (iii) poor efficiency, and (iv) damage to the introduced substance.

Microinjection is a technique using capillaries to physically inject substances into a cell. It is useful because it can selectively load cells with substances that are not compatible with other techniques, and it does not have the limitations and potential problems associated with many of the techniques described above. Microinjection is versatile in that practically any substance can be microinjected, even organelles. However, the extensive labor involved and very low throughput limits the usefulness of this method to specialized applications. What is needed is a high-throughput and versatile method of loading substances into cells.

Lasers have been used to introduce substances into cells, a process referred to as optoinjection. The optoinjection mechanism has been hypothesized to be a physical hole in the membrane caused by the laser when it is tightly focused on a portion of the cell's membrane. A limitation of optoinjection is the need to locate, and target with a laser, every single cell to be loaded. A related technique, termed optoporation, focuses the laser on the culture substrate, and the resulting shock wave causes a temporary permeabilization of the membranes of nearby cells. The disadvantages of optoporation are that significant cell death occurs, and cells at varying distances from the shock wave are loaded to different extents.

Thus, there is a need for a method and apparatus for rapid and efficient loading of a variety of substances into cells, with high cell survival rates. The present invention satisfies this need and provides related advantages, as well.

### SUMMARY OF THE INVENTION

The present invention provides methods for transiently permeabilizing a substantially stationary cell located within a volume defined by an effective distance from a solid surface, without prior knowledge of the specific three-dimensional location of the cell within that volume. The general method comprises irradiating such a cell with electromagnetic radiation that is sufficient to induce permeabilization of the cell membrane by directing the electromagnetic radiation towards the volume where the cell exists. A high rate of cell permeabilization can be attained by placing a large quantity of cells within a region of space that is within an effective distance from a solid surface, and then rapidly irradiating such a region in space with electromagnetic radiation. A high yield of permeabilization can be attained simultaneously with a high cell survival rate by selecting the proper combination of electromagnetic radiation dose parameters: wavelength, power density, and total exposure time. Energy density is a function of power density and total exposure time. In an electromagnetic radiation protocol wherein the radiation is administered in a series of pulses, total exposure time is a function of pulse duration and total number of pulses. Additionally, in electromagnetic radiation protocols wherein the radiation is administered as a series of pulses, the time between pulses (i.e., the periodicity of the pulses) may also be a critical parameter. Wherein during the permeabilized state the cells contact an aqueous medium that contains a substance that is to be loaded into the cells, the resulting methods provide rapid and efficient loading of a variety of substances into cells, with high cell survival rates.

Embodiments relate to apparatuses for transiently permeabilizing a substantially stationary cell located within a defined volume, without prior knowledge of the specific three-dimensional location of the cell within the defined volume. Generally, embodiments can include an apparatus that includes an energy source that emits electromagnetic radiation sufficient to induce permeabilization of a membrane of a cell, a directing device configured to direct the electromagnetic radiation to substantially the entirety of the defined volume in which the cell exists, and a solid surface, wherein the defined volume is partly bounded by the solid surface and further bounded by an effective distance from the solid surface. The solid surface can further include a substantially transparent material that participates in the optical path of the electromagnetic radiation. Embodiments can also include an apparatus that includes an energy source that emits electromagnetic radiation sufficient to induce permeabilization of a membrane of the cell, commands for directing the electromagnetic radiation to substantially the entirety of the defined volume in which the cell exists, and a directing device configured to direct the electromagnetic radiation in response to the commands.

Some embodiments relate to methods of transiently permeabilizing one or more cells. The methods can include a) maintaining the one or more cells in a substantially stationary position within an effective distance from a solid surface; and b) directing to the solid surface electromagnetic radiation sufficient to induce transient permeabilization of a membrane of the one or more cells, without prior knowledge of the specific three-dimensional location of the one or more cells, wherein the one or more cells can be coincident with the path of the electromagnetic radiation.

The electromagnetic radiation can have an energy density at the solid surface, for example, of at most about 0.001, 0.002, 0.003, 0.006, 0.01, 0.02, 0.03, 0.06, 0.1, 0.2, 0.3, 0.6, 1, 2, 3, 4, 5 and 6 μJ/μm². The electromagnetic radiation can have an energy density of any subset of the above densities individually or in any combination, and any range of the above densities. Furthermore, the electromagnetic radiation can have an energy density at the solid surface of about 0.001 to about 0.3 μJ/μm².

The effective distance can be, for example, less than about 1000 μm, 600 μm, 300 μm, 200 μm, 100 μm, 60 μm, 30 μm, 20 μm, 10 μm, 6 μm, 3 μm, 2 μm, 1 μm, and the like. The effective distance can be any subset of the above distances individually or in any combination, and any range of the above distances. Furthermore, the effective distance can be between about 1 μm to about 20 μm.

The electromagnetic radiation can be directed to the one or more cells for a period of time which can be, for example, at most on the order of about 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond. The period of time can be any subset of the above times individually or in any combination, and any range of the above times. Also, the one or more cells can be exposed to the electromagnetic radiation for a period of time of about 100 picoseconds to about 10 seconds, for example.

The electromagnetic radiation can have a wavelength, for example, between about 300 nanometers and about 3,000 nanometers, between about 330 nanometers and about 1,100 nanometers, between about 400 nanometers and about 700 nanometers, and the like.

The directing can include delivering a pulse of radiation to the solid surface, passing a beam of radiation across the solid surface according to a path pattern, and the like, for example.

The induction of transient permeabilization of a membrane in the one or more cells at can occur, for example, at a rate of at least 10, 30, 100, 300, 1000, 3000, 10,000, 30,000, 100,000, 300,000, 1,000,000, 3,000,000, 10,000,000, 30,000,000, 100,000,000 and 240,000,000 cells per second. The rate of induction of transient permeabilization can be any subset of the above rates individually or in any combination, and any range of the above rates. The method further can include inducing transient permeabilization in a membrane of the one or more cells at a rate of between about 300 to about 10,000,000 cells per second.

The probability of viability of the one or more cells after the transient permeabilizing of a membrane can be maintained, for example, at a value of at least 50%, 60%, 70%, 80%, 90%, 95%, 96%, 97%, 98% and 99%. The viability can be any subset of the above viability rates individually or in any combination, and any range of the above viability rates. Furthermore, the probability of viability of the one or more cells after the permeabilizing can be maintained at a value of at least 50% to at least 90%, for example.

The methods further can include contacting the one or more cells with a non-isotonic aqueous medium, for example. The methods can include contacting the one or more cells with an aqueous medium that contains a substance at a concentration lower than the concentration of the substance within the one more cells, such that the substance within the one or more cells can exit the one or more cells through a permeabilized membrane. Of course, one example of an aqueous medium concentration of substance that is lower than the concentration of substance within the one or more cells can be a zero concentration. The substance can be, for example, an ion, an organic molecule, an inorganic molecule, a colloidal particle, a polysaccharide, a peptide, a protein, a nucleic acid, a modified nucleic acid, and the like. Also, the one or more cells can contact an aqueous medium such that a substance within the aqueous medium can pass through the transiently permeabilized membrane of the cell to enter the cell. The transiently permeabilized membrane can recover to a substantially non-permeabilized state within a period of time, for example, of at most about 0.3 millisecond, 1 millisecond, 3 milliseconds, 10 milliseconds, 30 milliseconds, 100 milliseconds, 300 milliseconds, 1 second, 3 seconds, 10 seconds, 30 seconds, 1 minute, 2 minutes, 3 minutes, 6 minutes, 10 minutes, 20 minutes and 30 minutes. Also, the period of time can be any subset of the above time periods individually or in any combination, and any range of the above time periods. The transiently permeabilized membrane can recover to a substantially non-permeabilized state within a period of time of about 1 second to about 1 minute, for example.

The electromagnetic radiation can be directed to an area of the solid surface at a rate, for example, of at least about 0.0001, 0.0003, 0.001, 0.003, 0.01, 0.03, 0.1, 0.3, 1, 3, 10, 30, 100, 200, 300 and 400 square centimeters per second. The areas can be any subset of the above areas individually or in any combination, and any range of the above areas. Furthermore, the electromagnetic radiation can be directed to an area of the solid surface at a rate of about 0.0003 to about 10 square centimeters per second, for example.

The directing can include delivering two or more pulses of radiation to the solid surface at a rate, for example, of at least 1, 10, 100, 10³, 10⁴, 10⁵, 10⁶, 10⁷, 10⁸, and 10⁹ Hz. The rate can be any subset of the above rates individually or in any combination, and any range of the above rates. Furthermore, the directing can include delivering two or more pulses of radiation to the solid surface at a rate of about 10² to about 10⁴ Hz, for example. Also, the directing can include delivering two or more pulses of electromagnetic radiation to the solid surface according to a pulse target pattern. Also, at least two pulses of electromagnetic radiation can be directed to a single pulse target within the pulse target pattern.

The electromagnetic radiation can originate from an energy source such as, for example, a continuous wave laser, a pulsed laser, a continuous lamp, a flashlamp, and the like.

An individual pulse of the pulses of electromagnetic radiation can have a duration, for example, of at most on the order of 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond. The duration can be any subset of the above durations individually or in any combination, and any range of the above durations. For example, an individual pulse of the pulses of electromagnetic radiation can have a duration from about 100 picoseconds to about 10 seconds.

The electromagnetic radiation can be directed to a defined area on the solid surface, and the defined area can have an area for example, of at least 0.0001, 0.0003, 0.001, 0.003, 0.01, 0.03, 0.1, 0.3, 1, 3, 10, 30, 100, 200, 300 and 400 square centimeters. The area can be any subset of the above areas individually or in any combination, and any range of the above areas. For example, the electromagnetic radiation can be directed to a defined area on the solid surface, and the defined area can be an area of about 0.0001 to about 10 square centimeters. Also, the electromagnetic radiation can be directed simultaneously to substantially the entirety of the defined area.

The path of the electromagnetic radiation can have a width, for example, of at least 10, 12, 14, 16, 18, 20, 25, 30, 35, 40, 50, 60, 70, 80, 90, 100, 300, 1×10³, 2×10³, 3×10³, 4×10³, 5×10³, 6×10³, 7×10³, 8×10³, 9×10³ and 1×10⁴ micrometers. Also, the width can be any subset of the above widths individually or in any combination, and any range of the above widths. For example, the path of the electromagnetic radiation can have a width of about 10 micrometers to about 1000 micrometers.

The solid surface can be substantially transparent to electromagnetic radiation, for example. Also, the solid surface can include a polymer material, a glass material, and the like, for example.

Further embodiments relate to apparatuses for transiently permeabilizing a cell. The apparatuses can include, for example, a) an energy source that emits electromagnetic radiation sufficient to induce permeabilization of a membrane of a cell, wherein the cell can be substantially stationary and contained within a defined volume, wherein the specific coordinates of the cell within the defined volume are unknown, and wherein the defined volume can be partly bounded by a solid surface; b) a directing device configured to direct the electromagnetic radiation to substantially the entirety of the defined volume, wherein the cell can be coincident with the path of the electromagnetic radiation, and wherein the electromagnetic radiation within the defined volume can have an energy density at the solid surface of at most about 6 μJ/μm²; and optionally, c) the solid surface.

The electromagnetic radiation within the defined volume can have an energy density at the solid surface, for example, of at most about 0.001, 0.002, 0.003, 0.006, 0.01, 0.02, 0.03, 0.06, 0.1, 0.2, 0.3, 0.6, 1, 2, 3, 4 and 5 μJ/μm². Also, the energy density can be any subset of the above densities individually or in any combination, and any range of the above densities. For example, the electromagnetic radiation within the defined volume can have an energy density at the solid surface of about 0.001 to about 0.3 μJ/μm².

The directing device can direct pulses of electromagnetic radiation to the defined volume according to a pulse target pattern, for example. Also, at least two pulses of electromagnetic radiation can be directed to a single pulse target within the pulse target pattern. An individual pulse of the pulses of electromagnetic radiation can have a duration, for example, of at most on the order of about 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond. Also, duration can be any subset of the above durations individually or in any combination, and any range of the above durations. For example, the duration can be about 10 seconds to about 100 picoseconds.

The path of the electromagnetic radiation can have a width, for example, of at least about 10, 12, 14, 16, 18, 20, 25, 30, 35, 40, 50, 60, 70, 80, 90, 100, 300, 1×10³, 2×10³, 3×10³, 4×10³, 5×10³, 6×10³, 7×10³, 8×10³, 9×10³ and 1×10⁴ micrometers. Also, the width can be any subset of the above widths individually or in any combination, and any range of the above widths. For example, the width can be about 10 micrometers to about 1000 micrometers.

Still further embodiments relate to apparatuses for transiently permeabilizing a cell. The apparatuses can include, for example, a) an energy source that emits electromagnetic radiation sufficient to induce permeabilization of a membrane of a cell, wherein the cell can be a substantially stationary cell contained within a defined volume, and wherein the specific coordinates of the cell within the defined volume are unknown; b) commands for directing the electromagnetic radiation to substantially the entirety of the defined volume; and c) a directing device configured to direct the electromagnetic radiation in response to the commands.

The commands can include, for example, commands for directing pulses of electromagnetic radiation according to a pulse target pattern. Also, at least two pulses of electromagnetic radiation are directed to a single pulse target within the pulse target pattern, for example. An individual pulse of the pulses of electromagnetic radiation can have a duration, for example, of at most on the order of about 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond. Also, duration can be any subset of the above durations individually or in any combination, and any range of the above durations. For example, an individual pulse of the pulses of electromagnetic radiation can have a duration of about 100 picoseconds to about 10 seconds.

The electromagnetic radiation within the defined volume can have an energy density at the solid surface, for example, of at most about 0.001, 0.002, 0.003, 0.006, 0.01, 0.02, 0.03, 0.06, 0.1, 0.2, 0.3, 0.6, 1, 2, 3, 4, 5 and 6 μJ/μm². Also, energy density can be any subset of the above densities individually or in any combination, and any range of the above densities. For example, the electromagnetic radiation within the defined volume can have an energy density at the solid surface of about 0.001 to about 0.3 μJ/μm².

Furthermore, an instantaneous path of the electromagnetic radiation within the defined volume can have a width, for example, of at least 10, 12, 14, 16, 18, 20, 25, 30, 35, 40, 50, 60, 70, 80, 90, 100, 300, 1×10³, 2×10³, 3×10³, 4×10³, 5×10³, 6×10³, 7×10³, 8×10³, 9×10³ and 1×10⁴ micrometers. Also, the width can be any subset of the above widths individually or in any combination, and any range of the above widths. For example, the width can be about 10 micrometers to about 1000 micrometers.

Further embodiments relate to apparatuses for transiently permeabilizing a cell. The apparatuses can include, for example, a) an energy source that emits electromagnetic radiation sufficient to induce permeabilization of a membrane of a cell, wherein the cell can be a substantially stationary cell contained within a defined volume, wherein the specific coordinates of the cell within the defined volume are unknown, and wherein the defined volume can be partly bounded by a solid surface; b) a directing device configured to direct pulses of the electromagnetic radiation to substantially the entirety of the defined volume according to a pulse target pattern; and optionally, c) the solid surface.

An individual pulse of the pulses of electromagnetic radiation can have a duration, for example, of at most on the order of about 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond. Also, the duration can be any subset of the above durations individually or in any combination, and any range of the above durations. For example, an individual pulse of the pulses of electromagnetic radiation can have a duration of about 100 picoseconds to about 10 seconds.

Furthermore, at least two pulses of electromagnetic radiation can be directed to a single pulse target within the pulse target pattern. An individual pulse of the pulses of electromagnetic radiation within the defined volume can have a width, for example, of at least about 10, 12, 14, 16, 18, 20, 25, 30, 35, 40, 50, 60, 70, 80, 90, 100, 300, 1×10³, 2×10³, 3×10³, 4×10³, 5×10³, 6×10³, 7×10³, 8×10³, 9×10³ and 1×10⁴ micrometers. Also, the width can be any subset of the above widths individually or in any combination, and any range of the above widths. For example, an individual pulse of the pulses of electromagnetic radiation within the defined volume can have a width of about 10 micrometers to about 1000 micrometers.

Other embodiments relate to apparatuses for transiently permeabilizing a cell. The apparatus can include, for example, a) an energy source that emits electromagnetic radiation sufficient to induce permeabilization of a membrane of a cell, wherein the cell can be a: substantially stationary cell contained within a defined volume, and wherein the specific coordinates of the cell within the defined volume are unknown; b) commands for directing pulses of the electromagnetic radiation to substantially the entirety of the defined volume according to a pulse target pattern; and c) a directing device configured to direct the electromagnetic radiation in response to the commands.

An individual pulse of the pulses of electromagnetic radiation can have a duration, for example, of at most on the order of about 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond. Also, the duration can be any subset of the above durations individually or in any combination, and any range of the above durations. For example, an individual pulse of the pulses of electromagnetic radiation can have a duration of about 100 picoseconds to about 10 seconds.

The at least two pulses of electromagnetic radiation can be directed to a single pulse target within the pulse target pattern. An individual pulse of the pulses of electromagnetic radiation within the defined volume can have a width, for example, of at least about 10, 12, 14, 16, 18, 20, 25, 30, 35, 40, 50, 60, 70, 80, 90, 100, 300, 1×10³, 2×10³, 3×10³, 4×10³, 5×10³, 6×10³, 7×10³, 8×10³, 9×10³ and 1×10⁴ micrometers. Also, the width can be any subset of the above widths individually or in any combination, and any range of the above widths. For example, an individual pulse of the pulses of electromagnetic radiation within the defined volume can have a width of about 10 micrometers to about 1000 micrometers.

Still further embodiments relate to methods of transiently permeabilizing a cell. The methods can include, for example, a) maintaining the cell in a substantially stationary position within a defined volume, wherein the defined volume can be partly bounded by a solid surface and further bounded by an effective distance from the solid surface; and b) directing to substantially the entirety of the defined volume electromagnetic radiation sufficient to transiently induce permeabilization of a membrane of the cell.

The electromagnetic radiation within the defined volume can have an energy density at the solid surface, for example, at most about 0.001, 0.002, 0.003, 0.006, 0.01, 0.02, 0.03, 0.06, 0.1, 0.2, 0.3, 0.6, 1, 2, 3, 4, 5 and 6 μJ/μm². Also, the energy density can be any subset of the above densities individually or in any combination, and any range of the above densities. For example, the electromagnetic radiation within the defined volume can have an energy density at the solid surface of about 0.001 to about 0.3 μJ/μm².

The effective distance can be, for example, less than about 1000 μm, 600 μm, 300 μm, 200 μm, 100 μm, 60 μm, 30 μm, 20 μm, 10 μm, 6 μm, 3 μm, 2 μm, and 1 μm. Also, the effective distance can be any subset of the above distances individually or in any combination, and any range of the above distances. For example, the effective distance can be about 1 μm to about 20 μm.

The cell can be exposed to the electromagnetic radiation for a period of time, for example, of at most on the order of about 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond. Also, the time period can be any subset of the above time periods individually or in any combination, and any range of the above time periods. For example, the cell can be exposed to the electromagnetic radiation for a period of time of about 100 picoseconds to about 10 seconds.

The directing can include, for example, delivering a pulse of radiation to the defined volume, also, passing a beam of radiation through the defined volume according to a path pattern.

The methods further can include inducing transient permeabilization in cells at a rate, for example, of at least 10, 30, 100, 300, 1000, 3000, 10,000, 30,000, 100,000, 300,000, 1,000,000, 3,000,000, 10,000,000, 30,000,000, 100,000,000 and 240,000,000 cells per second. Also, the rate can be any subset of the above rates individually or in any combination, and any range of the above rates. For example, transient permeabilization can be induced in cells at a rate of between about 300 to about 10,000,000 cells per second.

The probability of viability of the cell after the permeabilizing can be maintained, for example, at a value of at least about 50%, 60%, 70%, 80%, 90%, 95%, 96%, 97%, 98% and 99%. Also, the value can be any subset of the above values individually or in any combination, and any range of the above values. For example, the probability of viability of the cell after the permeabilizing can be maintained at a value of at least 50% to at least 90%.

The methods can further include contacting the cell with a non-isotonic aqueous medium. The methods can include an aqueous medium that contains a substance that can pass through a permeabilized membrane of the cell when in contact with the cell.

The substance can be, for example, an ion, an organic molecule, an inorganic molecule, a polysaccharide, a peptide, a protein, a colloidal particle, a nucleic acid, a modified nucleic acid, and the like. The substance can enter or leave the cell via a permeabilized membrane.

The transiently permeabilized membrane can recover to a substantially non-permeabilized state within a period of time, for example, of at most about 0.3 millisecond, 1 millisecond, 3 milliseconds, 10 milliseconds, 30 milliseconds, 100 milliseconds, 300 milliseconds, 1 second, 3 seconds, 10 seconds, 30 seconds, 1 minute, 2 minutes, 3 minutes, 6 minutes, 10 minutes, 20 minutes and 30 minutes. Also, the recovery period of time can be any subset of the above recovery periods individually or in any combination, and any range of the above recovery periods. For example, the transiently permeabilized membrane can recover to a substantially non-permeabilized state within a period of time of about 1 second to about 1 minute.

The electromagnetic radiation can be directed to an area of the solid surface at a rate, for example, of at least 0.0001, 0.0003, 0.001, 0.003, 0.01, 0.03, 0.1, 0.3, 1, 3, 10, 30, 100, 200, 300 and 400 square centimeters per second. Also, the rate can be any subset of the above rates individually or in any combination, and any range of the above rates. For example, the electromagnetic radiation can be directed to an area of the solid surface at a rate of about 0.0003 to about 10 square centimeters per second

The electromagnetic radiation can have a power density, for example, of less than about 6×10⁷, 3×10⁷, 2×10⁷, 1×10⁷, 6×10⁶, 3×10⁶, 2×10⁶, 1×10⁶, 6×10⁵, 3×10⁵, 2×10⁵, 1×10⁵, 6×10⁴, 3×10⁴, 2×10⁴, and 1×10⁴ W/cm² within the defined volume. Also, the power density can be any subset of the above densities individually or in any combination, and any range of the above densities.

The directing can include delivering two or more pulses of radiation to the defined volume at a rate, for example, of at least 1, 10, 100, 10³, 10⁴, 10⁵, 10⁶, 10⁷, 10⁸, and 10⁹ Hz. Also, the rate can be any subset of the above rates individually or in any combination, and any range of the above rates. For example, the directing can include delivering two or more pulses of radiation to the defined volume at a rate of about 10² to about 10⁴ Hz. The directing can include delivering two or more pulses of electromagnetic radiation to the defined volume according to a pulse target pattern.

The electromagnetic radiation can originate from an energy source, for example, of a continuous wave laser, a pulsed laser, a continuous lamp, a flashlamp, and the like.

An individual pulse of the pulses of electromagnetic radiation can have a duration selected from the group of at most on the order of 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond. Also, the duration can be any subset of the above durations individually or in any combination, and any range of the above durations. For example, an individual pulse of the pulses of electromagnetic radiation can have a duration of about 100 picoseconds to about 10 seconds. Also, at least two pulses of electromagnetic radiation are directed to a single pulse target within the pulse target pattern.

The electromagnetic radiation can be directed to a defined area on the solid surface, and the defined area can have an area, for example, of at least about 0.0001, 0.0003, 0.001, 0.003, 0.01, 0.03, 0.1, 0.3, 1, 3, 10, 30, 100, 200, 300 and 400 square centimeters. Also, the area can be any subset of the above areas individually or in any combination, and any range of the above areas. For example, the electromagnetic radiation can be directed to a defined area on the solid surface, and the defined area can have an area of about 0.0001 to about 10 square centimeters. Also, the electromagnetic radiation can be directed simultaneously to substantially the entirety of the defined volume.

The path of the electromagnetic radiation within the defined volume can have a width, for example, of at least about 10, 12, 14, 16, 18, 20, 25, 30, 35, 40, 50, 60, 70, 80, 90, 100, 300, 1×10³, 2×10³, 3×10³, 4×10³, 5×10³, 6×10³, 7×10³, 8×10³, 9×10³ and 1×10⁴ micrometers. Also, the width can be any subset of the above widths individually or in any combination, and any range of the above widths. For example, the path of the electromagnetic radiation within the defined volume can have a width of about 10 micrometers to about 1000 micrometers.

Also, embodiments relate to further apparatuses for transiently permeabilizing a cell. The apparatuses can include, for example, a) an energy source that emits electromagnetic radiation sufficient to induce permeabilization of a membrane of a cell, wherein the cell can be substantially stationary and contained within a defined volume, and wherein the specific coordinates of the cell within the defined volume are unknown; b) commands for directing the electromagnetic radiation to a plurality of locations within the defined volume without regard to the characteristics of the plurality of locations; and c) a directing device configured to direct the electromagnetic radiation in response to the commands.

Other embodiments relate to apparatuses for transiently permeabilizing a cell. The apparatuses can include, for example, a) an energy source that emits electromagnetic radiation sufficient to induce permeabilization of a membrane of a cell, wherein the cell can be substantially stationary and contained within a defined volume, and wherein the specific coordinates of the cell within the defined volume are unknown; b) commands for directing the electromagnetic radiation to a plurality of locations comprising substantially the entirety of the defined volume, and wherein the electromagnetic radiation within the defined volume can have an energy density at the solid surface of at most 6 μJ/μm²; and c) a directing device configured to direct the electromagnetic radiation in response to the commands.

Embodiments relate to systems with a memory that can include a set of instructions, such that when executed the computer performs the action comprising directing to a solid surface electromagnetic radiation sufficient to induce permeabilization of a membrane of a substantially stationary cell, without prior knowledge of the specific three-dimensional location of the cell, wherein the cell can be coincident with the path of the electromagnetic radiation.

The cells of any of the embodiments can be any cell, including eucaryotic and procaryotic cells, mammalian cells and non mammalian cells, stem cells, research animal cells, plant cells, bacteria, fungi, viruses, and the like.

Some embodiments of the present invention are described in the following paragraphs:
1. A method of transiently permeabilizing one or more cells, comprising:
   a) maintaining said one or more cells in a substantially stationary position within an effective distance from a solid surface; and
   b) directing to said solid surface electromagnetic radiation sufficient to induce permeabilization of a membrane of said one or more cells, without prior knowledge of the specific three-dimensional location of said one or more cells, wherein said one or more cells is coincident with the path of said electromagnetic radiation;
2. The method of paragraph 1, wherein said electromagnetic radiation has an energy density at said solid surface selected from the group consisting of at most about 0.001, 0.002, 0.003, 0.006, 0.01, 0.02, 0.03, 0.06, 0.1, 0.2, 0.3, 0.6, 1, 2, 3, 4, 5 and 6 μJ/μm²;
3. The method of paragraph 1, wherein said electromagnetic radiation has an energy density at said solid surface of about 0.001 to about 0.3 μJ/μm²;
4. The method of paragraph 1, wherein said effective distance is selected from the group consisting of less than about 1000 μm, 600 μm, 300 μm, 200 μm, 100 μm, 60 μm, 30 μm, 20 μm, 10 μm, 6 μm, 3 μm, 2 μm, and 1 μm;
5. The method of paragraph 1, wherein said effective distance is between about 1 μm to about 20 μm;
6. The method of paragraph 1, wherein said electromagnetic radiation is directed to said one or more cells for a period of time selected from the group of at most on the order of 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond;
7. The method of paragraph 1, wherein said one or more cells are exposed to said electromagnetic radiation for a period of time of about 100 picoseconds to about 10 seconds;
8. The method of paragraph 1, wherein said directing comprises delivering a pulse of radiation to said solid surface;
9. The method of paragraph 1, wherein said directing comprises passing a beam of radiation across said solid surface according to a path pattern;
10. The method of paragraph 1, further comprising inducing permeabilization of a membrane in said one or more cells at a rate that is selected from the group of at least 10, 30, 100, 300, 1000, 3000, 10,000, 30,000, 100,000, 300,000, 1,000,000, 3,000,000, 10,000,000, 30,000,000, 100,000,000 and 240,000,000 cells per second;
11. The method of paragraph 1, further comprising inducing permeabilization in a membrane of said one or more cells at a rate of between about 300 to about 10,000,000 cells per second;
12. The method of paragraph 1, wherein the probability of viability of said one or more cells after said permeabilizing of a membrane of said one or more cells is maintained at a value selected from the group consisting of at least 50%, 60%, 70%, 80%, 90%, 95%, 96%, 97%, 98% and 99%;
13. The method of paragraph 1, wherein the probability of viability of said one or more cells after said permeabilizing is maintained at a value of at least 50% to at least 90%;
14. The method of paragraph 1, further comprising contacting said one or more cells with a non-isotonic aqueous medium;
15. The method of paragraph 1, further wherein said one or more cells contacts an aqueous medium such that a substance within said permeabilized membrane can pass through said permeabilized membrane;
16. The method of paragraph 1, further wherein said one or more cells contacts an aqueous medium that contains a substance that can exit said one or more cells through a permeabilized membrane;
17. The method of paragraph 16, wherein said substance is selected from the group consisting of an ion, an organic molecule, an inorganic molecule, and a colloidal particle;
18. The method of paragraph 16, wherein said substance is selected from the group consisting of a polysaccharide, a peptide, a protein, a nucleic acid, and a modified nucleic acid;
19. The method of paragraph 16, wherein said substance enters said one or more cells via a permeabilized membrane;
20. The method of paragraph 19, further wherein said permeabilized membrane recovers to a substantially non-permeabilized state within a period of time selected from the group consisting of at most about 0.3 millisecond, 1 millisecond, 3 milliseconds, 10 milliseconds, 30 milliseconds, 100 milliseconds, 300 milliseconds, 1 second, 3 seconds, 10 seconds, 30 seconds, 1 minute, 2 minutes, 3 minutes, 6 minutes, 10 minutes, 20 minutes and 30 minutes;
21. The method of paragraph 19, wherein said permeabilized membrane recovers to a substantially non-permeabilized state within a period of time of about 1 second to about 1 minute;
22. The method of paragraph 1, wherein said electromagnetic radiation is directed to an area of said solid surface at a rate that is selected from the group consisting of at least about 0.0001, 0.0003, 0.001, 0.003, 0.01, 0.03, 0.1, 0.3, 1, 3, 10, 30, 100, 200, 300 and 400 square centimeters per second;
23. The method of paragraph 1, wherein said electromagnetic radiation is directed to an area of said solid surface at a rate of about 0.0003 to about 10 square centimeters per second;
24. The method of paragraph 1, wherein said electromagnetic radiation has a power density of less than about 1×10⁴ W/cm² at said solid surface;
25. The method of paragraph 1, wherein said electromagnetic radiation has a power density of about 1×10⁴ W/cm² to about 6×10⁷ W/cm² at said solid surface;
26. The method of paragraph 1, wherein said directing comprises delivering two or more pulses of radiation to said solid surface at a rate selected from the group of at least 1, 10, 100, 10, 104, 10⁵, 10⁶, 10⁷, 10⁸, and 10⁹ Hz;
27. The method of paragraph 1, wherein said directing comprises delivering two or more pulses of radiation to said solid surface at a rate of about 10² to about 10⁴ Hz;
28. The method of paragraph 1, wherein said electromagnetic radiation originates from an energy source selected from the group consisting of a continuous wave laser, a pulsed laser, a continuous lamp, and a flashlamp;
29. The method of paragraph 1, wherein said directing comprises delivering two or more pulses of electromagnetic radiation to said solid surface according to a pulse target pattern;
30. The method of paragraph 29, wherein an individual pulse of said pulses of electromagnetic radiation has a duration selected from the group consisting of at most on the order of 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond;
31. The method of paragraph 29, wherein an individual pulse of said pulses of electromagnetic radiation has a duration from about 100 picoseconds to about 10 seconds;
32. The method of paragraph 29, wherein at least two pulses of electromagnetic radiation are directed to a single pulse target within said pulse target pattern;
33. The method of paragraph 1, wherein said electromagnetic radiation is directed to a defined area on said solid surface, and said defined area has an area selected from the group consisting of at least 0.0001, 0.0003, 0.001, 0.003, 0.01, 0.03, 0.1, 0.3, 1, 3, 10, 30, 100, 200, 300 and 400 square centimeters;
34. The method of paragraph 1, wherein said electromagnetic radiation is directed to a defined area on said solid surface, and said defined area has an area of about 0.0001 to about 10 square centimeters;
35. The method of paragraph 1, wherein said electromagnetic radiation is directed simultaneously to substantially the entirety of said defined area;
36. The method of paragraph 1, wherein said path of said electromagnetic radiation has a width selected from the group of at least 10, 12, 14, 16, 18, 20, 25, 30, 35, 40, 50, 60, 70, 80, 90, 100, 300, 1×10³, 2×10³, 3×10³, 4×10³, 5×10³, 6×10³, 7×10³, 8×10³, 9×10³ and 1×10⁴ micrometers;
37. The method of paragraph 1, wherein said path of said electromagnetic radiation has a width of about 10 micrometers to about 1000 micrometers;
38. An apparatus for transiently permeabilizing a cell, comprising:
   a) an energy source that emits electromagnetic radiation sufficient to induce permeabilization of a membrane of a cell, wherein said cell is substantially stationary and contained within a defined volume, wherein the specific coordinates of said cell within said defined volume are unknown, and wherein said defined volume is partly bounded by a solid surface;
   b) a directing device configured to direct said electromagnetic radiation to substantially the entirety of said defined volume, wherein said cell is coincident with the path of said electromagnetic radiation, and wherein said electromagnetic radiation within said defined volume has an energy density at said solid surface of at most about 6 μJ/μm²; and
   c) said solid surface;
39. The apparatus of paragraph 38, wherein said electromagnetic radiation within said defined volume has an energy density at said solid surface selected from the group consisting of at most about 0.001, 0.002, 0.003, 0.006, 0.01, 0.02, 0.03, 0.06, 0.1, 0.2, 0.3, 0.6, 1, 2, 3, 4 and 5 μJ/μm²;
40. The apparatus of paragraph 38, wherein said electromagnetic radiation within said defined volume has an energy density at said solid surface of about 0.001 to about 0.3 μJ/μm²;
41. The apparatus of paragraph 38, wherein said directing device directs pulses of electromagnetic radiation to said defined volume according to a pulse target pattern;
42. The apparatus of paragraph 40, wherein an individual pulse of said pulses of electromagnetic radiation has a duration selected from the group consisting of at most on the order of 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond;
43. The apparatus of paragraph 40, wherein an individual pulse of said pulses of electromagnetic radiation has a duration of about 10 seconds to about 100 picoseconds;
44. The apparatus of paragraph 40, wherein at least two pulses of electromagnetic radiation are directed to a single pulse target within said pulse target pattern;
45. The apparatus of paragraph 38, wherein said path of said electromagnetic radiation has a width selected from the group consisting of at least 10, 12, 14, 16, 18, 20, 25, 30, 35, 40, 50, 60, 70, 80, 90, 100, 300, 1×10³, 2×10³, 3×10³, 4×10³, 5×10³, 6×10³, 7×10³, 8×10³, 9×10³ and 1×10⁴ micrometers;
46. The apparatus of paragraph 38, wherein said path of said electromagnetic radiation has a width of about 10 micrometers to about 1000 micrometers;
47. An apparatus for transiently permeabilizing a cell, comprising:
   a) an energy source that emits electromagnetic radiation sufficient to induce permeabilization of a membrane of a cell, wherein said cell is a substantially stationary cell contained within a defined volume, and wherein the specific coordinates of said cell within said defined volume are unknown;
   b) commands for directing said electromagnetic radiation to substantially the entirety of said defined volume; and
   c) a directing device configured to direct said electromagnetic radiation in response to said commands;
48. The apparatus of paragraph 47, wherein said commands comprise commands for directing pulses of electromagnetic radiation according to a pulse target pattern;
49. The apparatus of paragraph 48, wherein an individual pulse of said pulses of electromagnetic radiation has a duration selected from the group consisting of at most on the order of about 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond;
50. The apparatus of paragraph 48, wherein an individual pulse of said pulses of electromagnetic radiation has a duration of about 100 picoseconds to about 10 seconds;
51. The apparatus of paragraph 48, wherein at least two pulses of electromagnetic radiation are directed to a single pulse target within said pulse target pattern;
52. The apparatus of paragraph 47, wherein said electromagnetic radiation within said defined volume has an energy density at said solid surface selected from the group consisting of at most about 0.001, 0.002, 0.003, 0.006, 0.01, 0.02, 0.03, 0.06, 0.1, 0.2, 0.3, 0.6, 1, 2, 3, 4, 5 and 6 μJ/μm²;
53. The apparatus of paragraph 47, wherein said electromagnetic radiation within said defined volume has an energy density at said solid surface of about 0.001 to about 0.3 μJ/μm²;
54. The apparatus of paragraph 47, wherein an instantaneous path of said electromagnetic radiation within said defined volume has a width selected from the group of at least 10, 12, 14, 16, 18, 20, 25, 30, 35, 40, 50, 60, 70, 80, 90, 100, 300, 1×10³, 2×10³, 3×10³, 4×10³, 5×10³, 6×10³, 7×10³, 8×10³, 9×10³ and 1×10⁴ micrometers;
55. The apparatus of paragraph 47, wherein an instantaneous path of said electromagnetic radiation within said defined volume has a width of about 10 micrometers to about 1000 micrometers;
56. An apparatus for transiently permeabilizing a cell, comprising:
   a) an energy source that emits electromagnetic radiation sufficient to induce permeabilization of a membrane of a cell, wherein the cell is a substantially stationary cell contained within a defined volume, wherein the specific coordinates of said cell within said defined volume are unknown, and wherein said defined volume is partly bounded by a solid surface;
   b) a directing device configured to direct pulses of said electromagnetic radiation to substantially the entirety of said defined volume according to a pulse target pattern; and
   c) said solid surface;
57. The apparatus of paragraph 56, wherein an individual pulse of said pulses of electromagnetic radiation has a duration selected from the group consisting of at most on the order of about 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond;
58. The apparatus of paragraph 56, wherein an individual pulse of said pulses of electromagnetic radiation has a duration of about 100 picoseconds to about 10 seconds;
59. The apparatus of paragraph 56, wherein at least two pulses of electromagnetic radiation are directed to a single pulse target within said pulse target pattern;
60. The apparatus of paragraph 56, wherein an individual pulse of said pulses of electromagnetic radiation within said defined volume has a width selected from the group consisting of at least about 10, 12, 14, 16, 18, 20, 25, 30, 35, 40, 50, 60, 70, 80, 90, 100, 300, 1×10³, 2×10³, 3×10³, 4×10³, 5×10³, 6×10³, 7×10³, 8×10³, 9×10³ and 1×10⁴ micrometers;
61. The apparatus of paragraph 56, wherein an individual pulse of said pulses of electromagnetic radiation within said defined volume has a width of about 10 micrometers to about 1000 micrometers;
62. An apparatus for transiently permeabilizing a cell, comprising:
   a) an energy source that emits electromagnetic radiation sufficient to induce permeabilization of a membrane of a cell, wherein said cell is a: substantially stationary cell contained within a defined volume, and wherein the specific coordinates of said cell within said defined volume are unknown;
   b) commands for directing pulses of said electromagnetic radiation to substantially the entirety of said defined volume according to a pulse target pattern; and
   c) a directing device configured to direct said electromagnetic radiation in response to said commands;
63. The apparatus of paragraph 62, wherein an individual pulse of said pulses of electromagnetic radiation has a duration selected from the group consisting of at most on the order of about 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond;
64. The apparatus of paragraph 62, wherein an individual pulse of said pulses of electromagnetic radiation has a duration of about 100 picoseconds to about 10 seconds;
65. The apparatus of paragraph 62, wherein at least two pulses of electromagnetic radiation are directed to a single pulse target within said pulse target pattern;
66. The apparatus of paragraph 62, wherein an individual pulse of said pulses of electromagnetic radiation within said defined volume has a width selected from the group consisting of at least about 10, 12, 14, 16, 18, 20, 25, 30, 35, 40, 50, 60, 70, 80, 90, 100, 300, 1×10³, 2×10³, 3×10³, 4×10³, 5×10³, 6×10³, 7×10³, 8×10³, 9×10³ and 1×10⁴ micrometers;
67. The apparatus of paragraph 62, wherein an individual pulse of said pulses of electromagnetic radiation within said defined volume has a width of about 10 micrometers to about 1000 micrometers;
68. A method of transiently permeabilizing a cell, comprising: a) maintaining said cell in a substantially stationary position within a defined volume, wherein said defined volume is partly bounded by a solid surface and further bounded by an effective distance from said solid surface; and b) directing to substantially the entirety of said defined volume electromagnetic radiation sufficient to transiently induce permeabilization of a membrane of said cell;
69. The method of paragraph 68, wherein said electromagnetic radiation within said defined volume has an energy density at said solid surface selected from the group of at most 0.001, 0.002, 0.003, 0.006, 0.01, 0.02, 0.03, 0.06, 0.1, 0.2, 0.3, 0.6, 1, 2, 3, 4, 5 and 6 μJ/μm²;
70. The method of paragraph 68, wherein said electromagnetic radiation within said defined volume has an energy density at said solid surface of about 0.001 to about 0.3 μJ/μm²;
71. The method of paragraph 68, wherein said effective distance is selected from the group of less than 1000 μm, 600 μm, 300 μm, 200 μm, 100 μm, 60 μm, 30 μm, 20 μm, 10 μm, 6 μm, 3 μm, 2 μm, and 1 μm;
72. The method of paragraph 68, wherein said effective distance is about 1 μm to about 20 μm;
73. The method of paragraph 68, wherein said cell is exposed to said electromagnetic radiation for a period of time selected from the group of at most on the order of 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond;
74. The method of paragraph 68, wherein said cell is exposed to said electromagnetic radiation for a period of time of about 100 picoseconds to about 10 seconds;
75. The method of paragraph 68, wherein said directing comprises delivering a pulse of radiation to said defined volume;
76. The method of paragraph 68, wherein said directing comprises passing a beam of radiation through said defined volume according to a path pattern;
77. The method of paragraph 68, further comprising inducing permeabilization in cells at a rate that is selected from the group of at least 10, 30, 100, 300, 1000, 3000, 10,000, 30,000, 100,000, 300,000, 1,000,000, 3,000,000, 10,000,000, 30,000,000, 100,000,000 and 240,000,000 cells per second;
78. The method of paragraph 68, further comprising inducing permeabilization in cells at a rate of between about 300 to about 10,000,000 cells per second;
79. The method of paragraph 68, wherein the probability of viability of said cell after said permeabilizing is maintained at a value selected from the group of at least 50%, 60%, 70%, 80%, 90%, 95%, 96%, 97%, 98% and 99%;
80. The method of paragraph 68, wherein the probability of viability of said cell after said permeabilizing is maintained at a value of at least 50% to at least 90%;
81. The method of paragraph 68, further comprising contacting said cell with a non-isotonic aqueous medium;
82. The method of paragraph 68, wherein said cell contacts an aqueous medium that contains a substance that can pass through a permeabilized membrane;
83. The method of paragraph 82, wherein said substance is selected from the group of ion, organic molecule, inorganic molecule, polysaccharide, peptide, protein, colloidal particle, nucleic acid, and modified nucleic acid;
84. The method of paragraph 82, wherein said substance enters said cell via a permeabilized membrane;
85. The method of paragraph 84, wherein said permeabilized membrane recovers to a substantially non-permeabilized state within a period of time selected from the group of at most 0.3 millisecond, 1 millisecond, 3 milliseconds, 10 milliseconds, 30 milliseconds, 100 milliseconds, 300 milliseconds, 1 second, 3 seconds, 10 seconds, 30 seconds, 1 minute, 2 minutes, 3 minutes, 6 minutes, 10 minutes, 20 minutes and 30 minutes;
86. The method of paragraph 84, wherein said permeabilized membrane recovers to a substantially non-permeabilized state within a period of time of about 1 second to about 1 minute;
87. The method of paragraph 68, wherein said electromagnetic radiation is directed to an area of said solid surface at a rate that is selected from the group of at least 0.0001, 0.0003, 0.001, 0.003, 0.01, 0.03, 0.1, 0.3, 1, 3, 10, 30, 100, 200, 300 and 400 square centimeters per second;
88. The method of paragraph 68, wherein said electromagnetic radiation is directed to an area of said solid surface at a rate of about 0.0003 to about 10 square centimeters per second;
89. The method of paragraph 68, wherein said electromagnetic radiation has a power density selected from the group of less than about 6×10⁷, 3×10⁷, 2×10⁷, 1×10⁷, 6×10⁶, 3×10⁶, 2×10⁶, 1×10⁶, 6×10⁵, 3×10⁵, 2×10⁵, 1×10⁵, 6×10⁴, 3×10⁴, 2×10⁴, and 1×10⁴ W/cm² within said defined volume;
90. The method of paragraph 68, wherein said directing comprises delivering two or more pulses of radiation to said defined volume at a rate selected from the group of at least 1, 10, 100, 10³, 10⁴, 10⁵, 10⁶, 10⁷, 10⁸, and 10⁹ Hz;
91. The method of paragraph 68, wherein said directing comprises delivering two or more pulses of radiation to said defined volume at a rate of about 10² to about 10⁴ Hz;
92. The method of paragraph 68, wherein said electromagnetic radiation originates from an energy source selected from the group of a continuous wave laser, a pulsed laser, a continuous lamp, and a flashlamp;
93. The method of paragraph 68, wherein said directing comprises delivering two or more pulses of electromagnetic radiation to said defined volume according to a pulse target pattern;
94. The method of paragraph 93, wherein an individual pulse of said pulses of electromagnetic radiation has a duration selected from the group of at most on the order of 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond;
95. The method of paragraph 93, wherein an individual pulse of said pulses of electromagnetic radiation has a duration of about 100 picoseconds to about 10 seconds;
96. The method of paragraph 93, wherein at least two pulses of electromagnetic radiation are directed to a single pulse target within said pulse target pattern;
97. The method of paragraph 68, wherein said electromagnetic radiation is directed to a defined area on said solid surface, and said defined area has an area selected from the group of at least 0.0001, 0.0003, 0.001, 0.003, 0.01, 0.03, 0.1, 0.3, 1, 3, 10, 30, 100, 200, 300 and 400 square centimeters;
98. The method of paragraph 68, wherein said electromagnetic radiation is directed to a defined area on said solid surface, and said defined area has an area of about 0.0001 to about 10 square centimeters;
99. The method of paragraph 68, wherein said electromagnetic radiation is directed simultaneously to substantially the entirety of said defined volume;
100. The method of paragraph 68, wherein the path of said electromagnetic radiation within said defined volume has a width selected from the group of at least 10, 12, 14, 16, 18, 20, 25, 30, 35, 40, 50, 60, 70, 80, 90, 100, 300, 1×10³, 2×10³, 3×10³, 4×10³, 5×10³, 6×10³, 7×10³, 8×10³, 9×10³ and 1×10⁴ micrometers;
101. The method of paragraph 68, wherein the path of said electromagnetic radiation within said defined volume has a width of about 10 micrometers to about 1000 micrometers;
102. An apparatus for transiently permeabilizing a cell, comprising:
   a) an energy source that emits electromagnetic radiation sufficient to induce permeabilization of a membrane of a cell, wherein said cell is substantially stationary and contained within a defined volume, and wherein the specific coordinates of said cell within said defined volume are unknown;
   b) commands for directing said electromagnetic radiation to a plurality of locations within said defined volume without regard to the characteristics of said plurality of locations; and
   a directing device configured to direct said electromagnetic radiation in response to said commands;
103. An apparatus for transiently permeabilizing a cell, comprising:
   a) an energy source that emits electromagnetic radiation sufficient to induce permeabilization of a membrane of a cell, wherein said cell is substantially stationary and contained within a defined volume, and wherein the specific coordinates of said cell within said defined volume are unknown;
   b) commands for directing said electromagnetic radiation to a plurality of locations comprising substantially the entirety of said defined volume, and wherein said electromagnetic radiation within said defined volume has an energy density at said solid surface of at most 6 μJ/μm²; and
   c) a directing device configured to direct said electromagnetic radiation in response to said commands;
104. A system with a memory comprising a set of instructions, such that when executed the computer performs the action comprising directing to a solid surface electromagnetic radiation sufficient to induce permeabilization of a membrane of a substantially stationary cell, without prior knowledge of the specific three-dimensional location of said cell, wherein said cell is coincident with the path of said electromagnetic radiation;
105. The method of claim 1, further wherein said one or more cells contacts an aqueous medium that lacks a substance, or contains the substance at a concentration lower than the concentration of the substance within said one more cells, such that said substance within said one or more cells can exit said one or more cells through a transiently permeabilized membrane;
106. The method of claim 105, wherein said substance is selected from the group consisting of an ion, an organic molecule, an inorganic molecule, a colloidal particle, a polysaccharide, a peptide, a protein, a nucleic acid, and a modified nucleic acid;
107. The method of claim 1, further wherein said one or more cells contacts an aqueous medium such that a substance within said aqueous medium can enter said one or more cells through a transiently permeabilized membrane;
108. The method of claim 107, wherein said substance is selected from the group consisting of an ion, an organic molecule, an inorganic molecule, a colloidal particle, a polysaccharide, a peptide, a protein, a nucleic acid, and a modified nucleic acid;
109. The method of claim 107, further wherein said transiently permeabilized membrane recovers to a substantially non-permeabilized state within a period of time selected from the group consisting of at most about 0.3 millisecond, 1 millisecond, 3 milliseconds, 10 milliseconds, 30 milliseconds, 100 milliseconds, 300 milliseconds, 1 second, 3 seconds, 10 seconds, 30 seconds, 1 minute, 2 minutes, 3 minutes, 6 minutes, 10 minutes, 20 minutes and 30 minutes;
110. The method of claim 107, wherein said transiently permeabilized membrane recovers to a substantially non-permeabilized state within a period of time of about 1 second to about 1 minute.

### BRIEF DESCRIPTION OF THE DRAWINGS

FIG. 1 is a perspective view of a defined volume (V, measuring x by y by d), depicting a defined area (A, measuring x by y) on a solid surface (S) and an effective distance (d) projected orthogonally away from the defined area. Also shown are several cells existing within the defined volume, and a substantially transparent solid material (M) forming the solid surface

FIG. 2 is a perspective view of one embodiment of a cell treatment apparatus and illustrates the outer design of the housing and display.

FIG. 3 is a perspective view of one embodiment of a cell treatment apparatus with the outer housing removed and the inner components illustrated.

FIG. 4 is a block diagram of the optical subassembly design within one embodiment of a cell treatment apparatus.

FIG. 5 is a perspective view of one embodiment of an optical subassembly within one embodiment of a cell treatment apparatus.

FIG. 6 is a side view of one embodiment of an optical subassembly that illustrates the arrangement of the scanning lens and the movable stage.

FIG. 7 is a bottom perspective view of one embodiment of an optical subassembly.

FIG. 8 is a top perspective view of the movable stage of the cell treatment apparatus.

FIG. 9—is a picture demonstrating silencing of GFP expression by optoinjection of DNA encoding siRNA, as determined by fluorescence after 48 hours.

FIG. 10 is a graph showing cell growth following optoinjection of siRNA into SU-DHL-6 cells.

FIG. 11 is a picture demonstrating loading of NIH-3T3 cells with Zn²⁺, as determined by fluorescence of the Zn²⁺-indicator RhodZin-1 after one minute.

### DETAILED DESCRIPTION

Embodiments are related to methods and apparatuses for nonspecifically irradiating cells with electromagnetic radiation for the purpose of inducing a transient state of permeability. The transient state of permeability is useful for permitting a variety of substances to enter into or to be loaded into cells (cell loading), or to depart from cells (cell unloading), while allowing the cells to recover to a substantially non-permeabilized state within a period of time that is conducive to the continued viability of the cells after loading/unloading.

A general discussion of optoinjection and optoporation methods is found in the following references, each of which is hereby incorporated herein by reference in its entirety: Guo, Y., Liang, H., & Berns, M. W. 1995. Laser-mediated gene transfer in rice. Physiol.Plant, 93: 19-24; Shirahata, Y., Ohkohchi, N., Itagak, H., & Satomi, S. 2001. New technique for gene transfection using laser irradiation. J. Invest.Med., 49: 184-190; Tao, W., Wilkinson, J., Stanbridge, E. J., & Berns, M. W. 1987. Direct gene transfer into human cultured cells facilitated by laser micropuncture of the cell membrane. PNAS, 84: 4180-4184; Tirlapur, U. K. & Konig, K. 2002. Targeted transfection by femtosecond laser. Nature, 418: 290-291; Kurata, S., Tsukakoshi, M., Kasuya, T., & Ikawa, Y. 1986. The laser method for efficient introduction of foreign DNA into cultured cells. Exp.Cell Res., 162: 372-378; Koller, M. R., Hanania, E. G., Eisfeld, T. M., & Palsson, B. O., U.S. Patent Application Publication No. 20020076744, published on Jun. 20, 2002 entitled “Optoinjection methods,” for U.S. patent application Ser. No. 09/961,691 filed Sep. 21, 2001; Palsson, B. O., U.S. patent application Ser. No. 10/359,483, filed Feb. 4, 2003, entitled “Method and Apparatus for Selectively Targeting Specific Cells within a Cell Population;” Krasieava, T. B., Chapman, C. F., LaMorte, V. J., Venugopalan, V., Berns, M. W., & Tromberg, B. J. 1998. Mechanisms of cell permeabilization by laser microirradiation. Proc.SPIE, 3260: 38-44; and Tsukakoshi, M., Kurata, S., Nominya, Y., Ikawa, Y., & Kasuya, T. 1984. A novel method of DNA transfection by laser microbeam cell surgery. Appl.Phys., 35: 135-140.

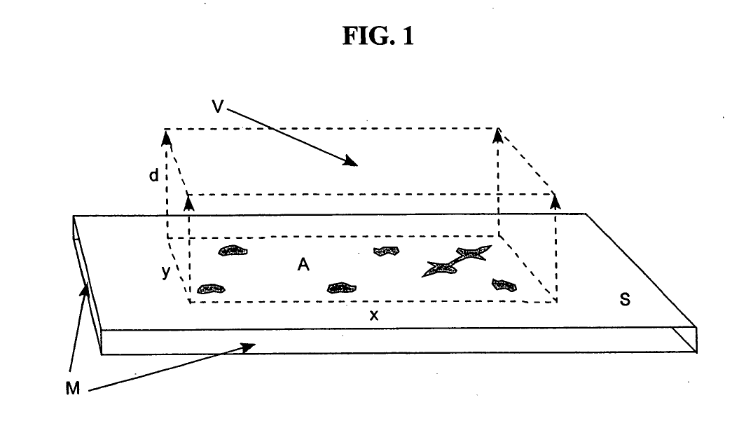

The methods and apparatuses described herein do not require knowledge of the specific three-dimensional locations of cells in order to induce a transient state of permeability. Instead, the cells to be transiently permeabilized can exist within a defined volume, wherein the defined volume has known dimensions and position in space. The cells preferably can be in a substantially stationary position within the defined volume, wherein substantially stationary means that the cells are not crossing the boundaries of the defined volume (either into or out from the defined volume) during the irradiation process. The defined volume is partly bounded by a defined area on a solid surface, wherein the defined area has known dimensions and position on the solid surface. The defined area can have a variety of useful sizes and/or boundaries (e.g., the area on the inside bottom surface of a single well of a multi-well tissue culture plate, or the area on the inside bottom surface of a tissue culture flask), depending upon the application of the invention, including but not limited to: at least 0.0001, 0.0003, 0.001, 0.003, 0.01, 0.03, 0.1, 0.3, 1, 3, 10, 30, 100, 200, 300 and 400 square centimeters. The defined volume further can be bounded by an effective distance projected orthogonally away from the defined area, wherein the effective distance is a predetermined distance within which the electromagnetic radiation is known to be effective for the purpose of inducing a transient state of permeability in a cell (FIG. 1). As used herein, “orthogonally” is the adverbial form of orthogonal which is defined as intersecting or lying at right angles. The transient state of permeability can be induced in a cell by directing to the defined volume electromagnetic radiation of a quality and quantity sufficient to induce permeabilization of the cell membrane. A cell that is contained within the irradiated defined volume is coincident with the path of the electromagnetic radiation, and such cell thereby receives a dose of electromagnetic radiation that induces permeabilization. When a cell is coincident with the path of electromagnetic radiation, it means that at least part of the cell and at least part of the electromagnetic radiation occupy a same region of space at the same moment.

In some embodiments the apparatuses can include an energy source that emits electromagnetic radiation sufficient to induce permeabilization of a cell membrane. General types of such energy sources include, but are not limited to: continuous wave lasers; pulsed lasers; continuous lamps; and flashlamps. Specific types of such energy sources include, but are not limited to: arc lamps (e.g., mercury, xenon, metal halide, etc.), with or without filters; light-emitting diodes (LEDs); dye lasers; gas lasers; solid-state lasers; Q-switched lasers, and the like. The path that the electromagnetic radiation takes through the defined volume at any one instant (i.e., the instantaneous path of the electromagnetic radiation within the defined volume) can have useful widths, for example, of at least 10, 12, 14, 16, 18, 20, 25, 30, 35, 40, 50, 60, 70, 80, 90, 100, 300, 1×10³, 2×10³, 3×10³, 4×10³, 5×10³, 6×10³, 7×10³, 8×10³, 9×10³ and 1×10⁴ micrometers. Specific width ranges within this group of widths can also be useful. For example, the width can be about 10 micrometers to about 1000 micrometers. Widths that are larger than a cell can be advantageous in that more than one cell can be irradiated simultaneously. In many embodiments, it is also useful to include optical elements in the optical path of the electromagnetic radiation (as it travels from the energy source to the defined volume) that shape the electromagnetic radiation into a beam that is within the useful range of beam width within the defined volume.

In some embodiments, the apparatuses can further include a directing device configured to direct electromagnetic radiation to the defined volume, such that the electromagnetic radiation is projected through substantially the entirety of the defined volume. Once the electromagnetic radiation leaves the energy source, it can require at least one element to direct it to the defined volume with a quality and quantity sufficient to induce permeabilization of the cell membrane. Examples of elements that direct the trajectory of the electromagnetic radiation from the energy source to the defined volume include, but are not limited to: a reflective element (such as a mirror), a refractive element (such as a prism or a fiber optic), a diffractive element, a galvanometric element, a piezo-electric tilt platform, and an acousto-optical deflector.

One example of directing the electromagnetic radiation to the defined volume with a sufficient quality is shaping the geometric profile of a beam of electromagnetic radiation with optical elements known in the art of optical design. The geometric profile can include, without limitation: the convergence/divergence angle within the defined volume, the diameter of the beam within the defined volume, the diameter of the beam waist within the defined volume, radial energy distribution within the beam, and the like. Another example of directing the electromagnetic radiation to the defined volume with a sufficient quality is controlling the duration of individual pulses of electromagnetic radiation, wherein it is useful for an individual pulse to have a duration (i.e., a pulse width) of between on the order of an attosecond and on the order of 1000 seconds. Such control can be accomplished, for example, via: mechanical shutters, optical shutters, electronic control of the energy source to generate discrete and defined pulses and the like. A still further example of directing the electromagnetic radiation to the defined volume with a sufficient quality is controlling the frequency of successive pulses directed to the same location within the defined volume (via, for example: mechanical shutters, optical shutters, electronic control of the energy source to generate discrete and defined pulses, and the like); examples of such pulse frequencies include, but are not limited to, frequencies in the range of at least 1 Hz to 10⁹ Hz. Also, an example of directing the electromagnetic radiation to the defined volume with a sufficient quality is controlling the wavelength of the electromagnetic radiation via one or more filters, for example.

An example of directing the electromagnetic radiation to the defined volume with a sufficient quantity is controlling the energy planar density, or the energy per unit area, that is projected towards the defined area on the solid surface, while projecting the electromagnetic radiation through substantially the entire defined volume. For example, this can include: concentrating the energy of a radiation beam within a defined beam diameter; shaping a laser beam waist by controlling the convergence angle of the laser beam within the defined volume; attenuating the energy of a radiation beam with filters; limiting the duration of radiation beam exposure within the defined volume by controlling the pulse width; limiting the cumulative duration of radiation beam exposure by controlling the number of pulses directed to a particular location within the defined area; or, directing a radiation beam, having a cross sectional area that is smaller than the defined area, to multiple locations within the defined area, such that the entire defined area receives a substantially uniform cumulative energy planar density. Furthermore, the radiation beam can be continuous (e.g., not flashed or pulsed) and also can have a cross sectional area smaller than the defined area. The entire defined volume can receive a substantially uniform cumulative energy density by passing the beam across the defined area in a path pattern that controls the amount of path overlap within acceptable limits of cumulative energy planar density, while projecting the electromagnetic radiation through substantially the entire defined volume. Researchers have permeabilized cells by localizing electromagnetic radiation to small portions of the cell membrane (with irradiation spots smaller than the cell diameter) and utilizing energy densities at or above 7μ (micro) J/μ (micro) m² (Tao, W., Wilkinson, J., Stanbridge, E. J., & Berns, M. W. 1987. Direct gene transfer into human cultured cells facilitated by laser micropuncture of the cell membrane. PNAS, 84: 4180-4184, at approximately 7 to 21 μJ/μm²; Palumbo G, Caruso M, Crescenzi E, Tecce M F, Roberti G, Colasanti A. 1996 Targeted gene transfer in eucaryotic cells by dye-assisted laser optoporation. J Photochem Photobiol B. 36(1):41-6; at approximately 1.6×10⁵ μJ/μm²; and Guo, Y., Liang, H., & Bern, M. W. 1995. Laser-mediated gene transfer in rice. Physiol.Plant, 93: 19-24, at approximately 2.5×10² to 1.3×10³ μJ/μm²; each of which is hereby incorporated herein by reference in its entirety). However, some, embodiments of the instant invention preferably can utilize energy densities at or below 6, 1, 0.1, 0.01, or even 0.001 μJ/μm² for transiently permeabilizing a cell, particularly when the entire cell is irradiated (as opposed to localizing the radiation to only a portion of the membrane), or when the energy is delivered within a very short period of time (such as within a period of time that is less than a microsecond, nanosecond, picosecond, femtosecond, or an attosecond).

To achieve an energy density effective for inducing transient permeabilization, it can be useful to expose cells to electromagnetic radiation for varying periods of time, depending upon the nature of the energy source and the power density achieved within the defined volume. For example, an energy source with a relatively low power output (e.g., a continuous lamp) might require on the order of 1000 seconds to induce a sufficient degree of transient permeabilization. As a further example, an energy source that delivers a pulse of very high power (e.g., a flashlamp or a pulsed laser) might require only a single pulse of radiation to induce a sufficient degree of transient permeabilization, enabling an exposure period on the order of a microsecond, nanosecond, picosecond, femtosecond, or even attosecond.

Another example of directing the electromagnetic radiation to the defined volume with a sufficient quantity is controlling the power planar density, or the power per unit area, that is projected towards the defined area on the solid surface while projecting the electromagnetic radiation through substantially the entire defined volume. For example, this can include: concentrating the power of a radiation beam within a defined beam diameter; shaping a laser beam waist by controlling the convergence angle of the laser beam within the defined volume; or, attenuating the power of a radiation beam with filters. Hence, directing can include more than just controlling the trajectory of the electromagnetic radiation within the defined volume, for it also can include controlling the quality and quantity of the electromagnetic radiation within the defined volume, depending on the nature of the energy source and the needs of the biological application.

A solid surface can be presented in a variety of cell-containment devices, including, but not limited to: standard tissue culture multiple-well plates (e.g., 6-well, 12-well, 24-well, 48-well, 96-well, 384-well, and 1536-well plates); petri dishes; microscope slides; plastic bags; tissue culture flasks; and tissue culture bottles. The solid surface of such cell-containment devices generally contacts an aqueous cell medium. In some embodiments, the optical path of the electromagnetic radiation, from the energy source to the defined volume, may or may not include the solid material that forms the solid surface, thereby enabling at least two broadly different types of optical path configurations, each with their unique advantages.

In one type of optical path configuration, the optical path approaches the defined volume from the side of the solid surface that includes the cells (i.e., the cellular space). One example of this type of optical path configuration is where the cells and the aqueous medium that the cells are in contact with are contained within a containment device, wherein the containment device has a solid floor oriented horizontally, solid walls, and either an open top or a top component that is substantially transparent to the electromagnetic radiation. The electromagnetic radiation approaches the defined volume within the containment device from above the containment device, passing through the open top or the substantially transparent top component, through the top liquid interface, through the aqueous medium, into the top boundary of the defined volume, through the defined volume, and to the defined area on the solid floor surface, thereby irradiating cells contained within the defined volume. In this type of optical path configuration, the solid material that forms the solid surface does not need to be transparent to the electromagnetic radiation incident upon the defined area, since the electromagnetic radiation has already passed through the defined volume at the time of incidence with the defined area. A distinct advantage of this type of optical path configuration is that the cell containment device can be simpler and cheaper to manufacture, since a special transparent material is not required for the solid material that forms the solid surface.

In another type of optical path configuration, the optical path approaches the defined volume from the side of the solid surface that includes the solid material. One example of this type of optical path configuration is where the cells and the aqueous medium that the cells are in contact with are contained within a containment device, wherein the containment device has walls and a horizontally oriented solid floor comprising a material that is substantially transparent to the electromagnetic radiation. The electromagnetic radiation approaches the defined volume within the containment device from below the containment device, passing through the bottom of the substantially transparent solid floor, through the solid floor, through the defined area on the solid floor surface, and through the defined volume, thereby irradiating cells contained within the defined volume. In this type of optical path configuration, the solid material that forms the solid surface must be substantially transparent to the electromagnetic radiation, since the electromagnetic radiation must pass through the solid material to reach the defined volume. Advantages of this type of optical path configuration include, but are not limited to: a) a containment device having a substantially transparent floor enables high-resolution imaging of the cells; and, b) the optical path through the aqueous cell medium is minimized, thereby minimizing variability associated with the optical properties of the aqueous phase. Furthermore, depending upon the application and the particular embodiment of the present invention, the solid surface can comprise a variety of materials, including, but not limited to, polymers and glass.

The apparatuses can also include commands for directing electromagnetic radiation to substantially the entirety of a defined volume. At any given instant, the electromagnetic radiation that is directed to the defined volume will intersect a portion of the defined volume, up to and including the entire defined volume. Such a portion of the defined volume that is intersected by the electromagnetic radiation at a given instant is herein called the volume of intersection. Wherein the volume of intersection is less than the entirety of the defined volume, the commands must direct the electromagnetic radiation to multiple volumes of intersection, or sweep the volume of intersection through a path in space, in order to ensure that the entirety of the defined volume is irradiated. Examples of commands useful for directing electromagnetic radiation to substantially the entirety of a defined volume include, but are not limited to: a command for directing electromagnetic radiation according to a pattern of pulse targets (i.e., a pulse target pattern) for individual pulses of electromagnetic radiation, wherein an individual pulse target may receive one, two, or more pulses; a command for directing electromagnetic radiation according to a grid pattern (such as, for instance, a 2-dimensional orthogonal grid pattern, or a 3-dimensional orthogonal grid pattern) of pulse targets for individual pulses of electromagnetic radiation; a command for directing electromagnetic radiation according to a sweeping pattern for a continuous beam of radiation, wherein the sweeping pattern allows for an amount of beam overlap that is from virtually 0% to 100%; a command for a single pulse of electromagnetic radiation; a command for multiple pulses of electromagnetic radiation; a command for a continuous beam of electromagnetic radiation to be directed to the defined volume for a certain period of time; a command for the energy source to turn on or start radiating; a command for the energy source to turn off or stop radiating; a command for a shutter to open or close; and, a command for a mirror or lens to move to a certain position. For instance, wherein the volume of intersection of an individual pulse of a pulsed laser energy source can comprise only 10% of the entire defined volume, the commands can direct the electromagnetic radiation to substantially the entirety of the defined volume by commanding a series of at least 10 laser pulses to take place, wherein each laser pulse has a unique volume of intersection, and the overlap volumes of the 10 unique volumes of intersection are minimized (e.g., at zero percent overlap, 10 pulses at 10% of the entire defined volume yields 100% of the entirety of the defined volume); such a series of 10 pulses can, for example, be directed to the defined volume according a pulse target pattern consisting of an orthogonal grid of 2 pulse targets by 5 pulse targets, wherein each pulse target creates a unique volume of intersection when the electromagnetic radiation is directed to that pulse target, and wherein the collection of 10 unique volumes of intersection includes substantially the entire defined volume. As a second instance, wherein the beam of electromagnetic radiation from a continuous laser energy source can comprise only 1% of the entire defined volume at any given instant, and about 1 millisecond of radiation exposure comprises the energy dose that is sufficient to induce permeabilization of a membrane of a cell within the defined volume, the commands can direct the electromagnetic radiation to substantially the entirety of the defined volume within a period of approximately 100 milliseconds by commanding the beam to sweep through the entirety of the defined volume according to a path pattern that includes a point-specific dwell time of about 1 millisecond and virtually no path overlap within the defined volume. For example, the commands can be generated automatically, via computer or other electronic control, or manually, via human operator control. It is useful for an apparatus featuring such commands to also feature a directing device configured to direct the electromagnetic radiation to the defined volume in response to such commands. Such directing device configured to direct the electromagnetic radiation can include, but are not limited to: a reflective element, a refractive element, a diffractive element, a galvanometric element, a piezo-electric tilt platform, an acousto-optical deflector, a shutter, and a filter. Such directing device configured to direct the electromagnetic radiation can further include electronic or mechanical actuators that are responsive to such commands.

A beam of electromagnetic radiation that intersects the defined volume at a volume of intersection that is smaller than the defined volume can require directing to multiple volumes of intersection, such that the entire defined volume receives a substantially uniform dose of electromagnetic radiation. Herein, a substantially uniform dose is understood to mean that the dose is substantially within a defined range of quantification values. The dose of electromagnetic radiation can be quantified in a variety of ways: energy planar density, power planar density, energy volumetric density, power volumetric density, energy flux, and other electromagnetic radiation quantification standards known in engineering and optical disciplines.

Some embodiments utilize a wavelength of electromagnetic radiation that can be significantly absorbed, diffused, refracted, or reflected by various materials and objects situated within the defined volumes. The materials and objects can include, but are not limited to, viable cells, nonviable cells, cell debris, and aqueous medium. The design of such embodiments can take into consideration the effects that the various materials and objects might have on the transmission of the electromagnetic radiation throughout the defined volume. Wavelengths of electromagnetic radiation that are useful for transiently permeabilizing a cell are approximately in the range of 300 nanometers to 3000 nanometers, with wavelengths in the visible part of the electromagnetic spectrum (approximately 400 nanometers to 700 nanometers) particularly useful. An example of a wavelength range that includes near-UV wavelengths useful for transiently permeabilizing a cell is the range of 330 nanometers to 400 nanometers. An example of a wavelength range that includes near-IR wavelengths useful for transiently permeabilizing a cell is the range of 700 nanometers to 1100 nanometers.

The undesirable impacts of optical phenomena such as absorption, diffusion, refraction or reflection within a defined volume can be countered by utilizing an electromagnetic radiation dosage sufficient to render insignificant such undesirable impacts. Also, the effective distance from the solid surface that bounds the defined volume can be limited, thereby limiting the defined volume within which cells can be located to a distance that is sufficiently effective for the induction of transient permeabilization of a cellular membrane, taking into consideration radiation diffusion, refraction, reflection and absorption. For example, the effective distance can range from the thickness of a single cell adhering to the solid surface (for example, on the order of 1μ (micro) m) up to on the order of 1000 μm. A distance beyond on the order of 1000 μm may experience a significant amount of attenuation of the energy or power density of the electromagnetic radiation. Other ways of countering the undesirable impacts include, but are not limited to: utilizing a lower power over a longer period of time, so as to maintain the desired total energy dosage while reducing temperature rise within the defined volume due to electromagnetic radiation absorption; and, utilizing multiple angles of incidence of the electromagnetic radiation at the defined surface.

The rate of cell permeabilization can be controlled in a variety of ways. The electromagnetic radiation dosage method and quantity can be designed for inducing permeabilization in a particular type of cell at a particular range of cell density within the defined volume. This design information can be used to choose or design an appropriate energy source and directing device to deliver the designed dosage. Cells can be placed within the defined volume at a wide range of cell densities, ranging for example, from a single cell per defined volume to cell densities found in living animal tissues (for example, up to 10⁸ cells per cubic centimeter). Additionally, if the energy source has a sufficient power output and the directing device can direct the electromagnetic radiation to a defined area at a rate of up to 400 square centimeters per second, then the rate of cell permeabilization is a function of the cell density within the defined volume and the rate of defined area irradiation (whereby the defined volume is substantially irradiated in the course of irradiating the defined area). An example of a high-throughput cell permeabilization apparatus is an apparatus that contains a flashlamp and a directing device so as to simultaneously irradiate in their entirety up to four standard multi-well plates, each measuring approximately 8.5 cm by 12.7 cm, with a single pulse of radiation from the flashlamp lasting less than one second, thereby irradiating over 400 square centimeters per second; in this example, the defined volume comprises all of the wells contained in all of the plates. For example, where each standard multi-well plate contains at least 60 million cells, over 240 million cells per second can be irradiated. Other apparatus and method embodiments can be created based on these principles, yielding a wide variety of useful cell permeabilization rates. In certain embodiments, irradiation of an area can proceed at a rate of at least 0.0001, 0.0003, 0.001, 0.003, 0.01, 0.03, 0.1, 0.3, 1, 3, 10, 30, 100, 200, 300 or 400 square centimeters per second, and more preferably at a rate of at least about 0.0003 to about 10 square centimeters per second. In certain embodiments, permeabilization of cells can proceed at a rate of at least 10, 30, 100, 300, 1000, 3000, 10,000, 30,000, 100,000, 300,000, 1,000,000, 3,000,000, 10,000,000, 30,000,000, 100,000,000 or 240,000,000 cells per second, and more specifically at a rate of at least about 300 to at least about 10,000,000 cells per second.

The transient state of permeability is useful for loading or unloading a variety of substances into cells, while allowing the cells to recover to a substantially non-permeabilized state within a period of time that is conducive to the continued viability of the cells after loading. Unloading for example, can occur when a substance is present within a cell membrane at a greater concentration that it is found outside of a cell membrane, the substance will exit the location with the higher concentration to the location with the lower concentration. To enable the loading of substances into transiently permeabilized cells, the substances can be contained in the aqueous medium that surrounds the cells within the defined volume. Such substances include, but are not limited to, ions, organic molecules, inorganic molecules (e.g., quantum dots (Han, M., Gao, X., Su, J. Z., & Nie, S. 2001. Quantum-dot-tagged microbeads for multiplexed optical coding of biomolecules. Nat.Biotech., 19: 631-635); which is incorporated herein by reference in its entirety), polysaccharides, peptides, proteins, colloidal particles, nucleic acids (e.g., oligonucleotides, polynucleotides, and plasmids) and modified nucleic acids (e.g., peptide nucleic acids). A nucleic acid can be single-stranded or double-stranded DNA or RNA, depending upon the application. An oligonucleotide is a sequence of up to about 20 nucleotides joined by phosphodiester bonds. Polynucleotides generally are sequences of more than about 20 nucleotides. Examples of ions include, without limitation, zinc and calcium ions. Examples of inorganic molecules include, without limitation, semiconductor nanocrystals (also known as quantuum dots). During the transient state of permeability, such a substance can enter the cell via the permeabilized membrane.

When the dosage of electromagnetic radiation is properly controlled, the permeabilized membrane can recover to a substantially non-permeabilized state within a period of time that is conducive to both loading a sufficient quantity of substance into the cell and the continued viability of the cell after loading. Useful periods of transient permeability can be as short as less than 0.3 millisecond for ions (Nilius B, Hess P, Lansman J B, Tsien R W). A novel type of cardiac calcium channel in ventricular cells. Nature. 1985 Aug. 1-7; 316 (6027): 443-6; which is incorporated herein by reference in its entirety) and for other small molecules. Periods of transient permeability beyond on the order of 30 minutes generally result in lower cell viability rates. Loaded substances may persist in their original forms for varying periods of time within the cell after the membrane returns to a substantially non-permeabilized state, depending upon the fate of the substance in the particular cell. For example, the substance may be rapidly hydrolyzed, phosphorylated, enzymatically cleaved, or incorporated into the cell's genome. It is generally desirable to adjust the dosage (quantity, quality, and method of administration) of the electromagnetic radiation such that the post-loading viability of the cells is maintained above at least 50%, 60%, 70%, 80%, 90%, 95%, 96%, 97%, 98% or 99%. Preferably, the viability is at least about 50% to at least about 90%.

Various points in time can be chosen to determine the post-loading viability of the cells, the suitability of which depends upon the specific application. For example, the point in time can be the time wherein it can be determined that a substance has entered the cell, the time wherein the substance impacts the cell metabolism or genetic circuitry, or a fixed post-loading period of time, such as 24 hours.

In some embodiments, to maintain acceptably high levels of cell viability, the electromagnetic radiation dosage generally has a power density of less than 1×10¹³ Watts(W)/cm². Tirlapur et al. (Nature, Vol. 418, 18 Jul. 2002, pp. 290-1) discloses use of an average power density of 10¹² W/cm² which equates to a peak density of 10¹⁹ W/cm² during a single pulse, Tao et al. (Proc. Natl. Acad. Sci. USA, Vol. 84, pp. 4180-4184, June 1987) at approximately 7.3×10¹⁰ to 2.1×10¹¹ W/cm², Palumbo et al. (J. of Photochem. Photobio. B: Biology 36, 1996, pp. 41-46) at approximately 6.4×10⁷ W/cm², and Guo et al. (Physiologia Plantarum 93, pp. 19-24, 1995) at approximately 1.7×10¹² to 8.5×10¹² W/cm². Tirlapur et al., Tao et al., and Palumbo et al. are each incorporated herein by reference in their entireties. In some embodiments, for example, if the exposure time is brief enough, or if the requirement for cell viability is relatively low, then power densities greater than about 1×10³, 2×10¹³, 3×10¹³, 6×10¹³, 1×10¹⁴, 2×10¹⁴, 3×10¹⁴, 6×10¹⁴, 1×10¹⁵, 2×10¹⁵, 3×10¹⁵, 6×10¹⁵, 1×10¹⁶, 2×10¹⁶, 3×10¹⁶, 6×10¹⁶, and 1×10¹⁷ W/cm² can be utilized. Additionally, in other embodiments for example, if the exposure time is long enough, and the area of irradiation is large enough to accommodate a feasible rate of cell permeabilization, then power densities less than about 6×10⁷, 3×10⁷, 2×10⁷, 1×10⁷, 6×10⁶, 3×10⁶, 2×10⁶, 1×10⁶, 6×10⁵, 3×10⁵, 2×10⁵, 1×10⁵, 6×10⁴, 3×10⁴, 2×10⁴, and 1×10⁴ W/cm² can be utilized.

Additional enhancement of the substance loading rate into cells can be achieved by utilizing non-isotonic aqueous media in conjunction with the induction of a transient state of permeability in the cells. The cells can be exposed to a non-isotonic aqueous medium before, during, or after electromagnetic radiation-induced permeabilization, in order to achieve an enhanced substance-loading rate. The rate can be expressed in various terms, including, but not limited to: quantity of substance loaded per cell; quantity of substance loaded per cell per unit time; or, fraction (or alternatively, percentage) of total cells within the defined volume that are successfully loaded with a threshold level of the substance. An example of a useful hypotonic aqueous medium is a solution consisting of 25 mM KCl, 0.3 mM KH₂PO₄, and 90 mOsm/Kg myoinositol; this solution can also be mixed in a 1:1 ratio with standard isotonic phosphate-buffered saline (PBS) solution to create a hypotonic medium of more moderate hypo-osmolarity. An example of a useful hypertonic aqueous medium is a solution consisting of 25 mM KCl, 0.3 mM KH₂PO₄, and 400 mOsm/Kg myoinositol; this solution can also be mixed in a 1:1 ratio with standard isotonic PBS solution to create a hypertonic medium of more moderate hyper-osmolarity. Those skilled in the art will recognize other non-isotonic aqueous media formulations that also can be useful for enhancing the substance loading rate.

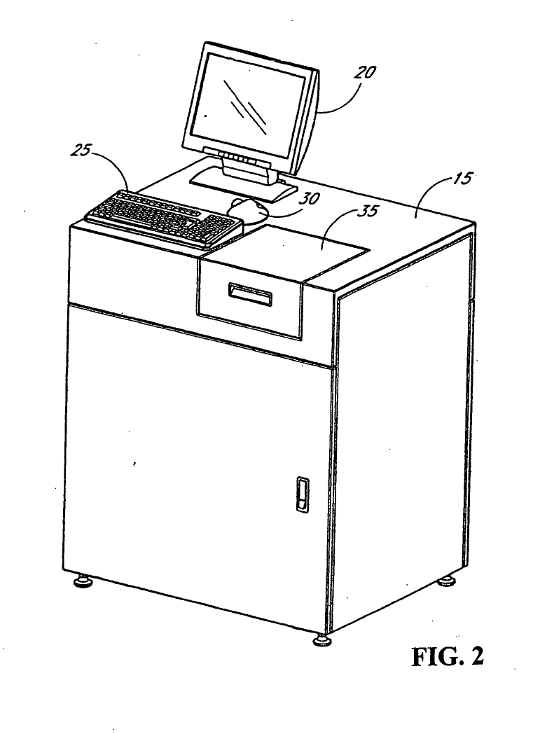

FIG. 2 is an illustration of one embodiment of an apparatus 10 that can be used to transiently permeabilize a cell and/or a load a cell with a substance. The apparatus 10 includes a housing 15 that stores the inner components. The housing includes laser safety interlocks to ensure safety of the user, and also limits interference by external influences (e.g., ambient light, dust, etc.). Located on the upper portion of the housing 15 is a display unit 20 for displaying process information. A keyboard 25 and mouse 30 are used to input data and control the apparatus 10. An access door 35 provides access to a movable stage that holds a container of cells.

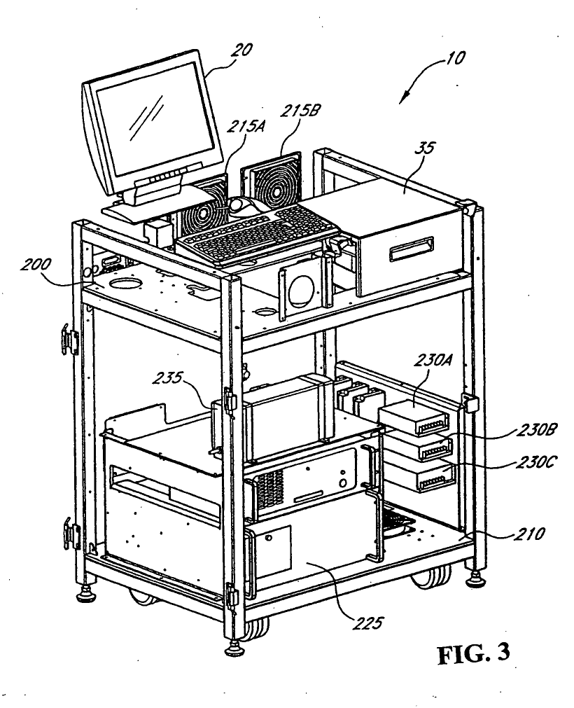

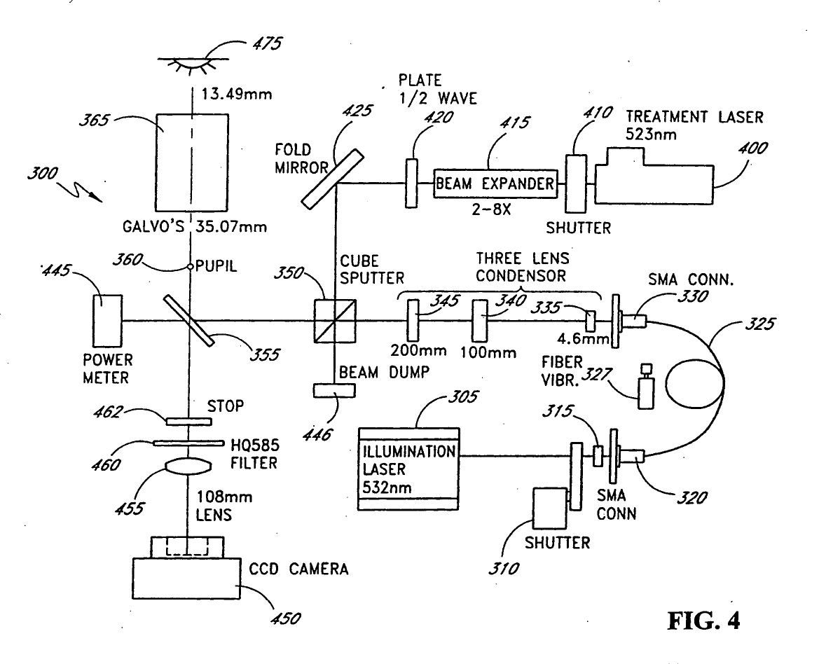

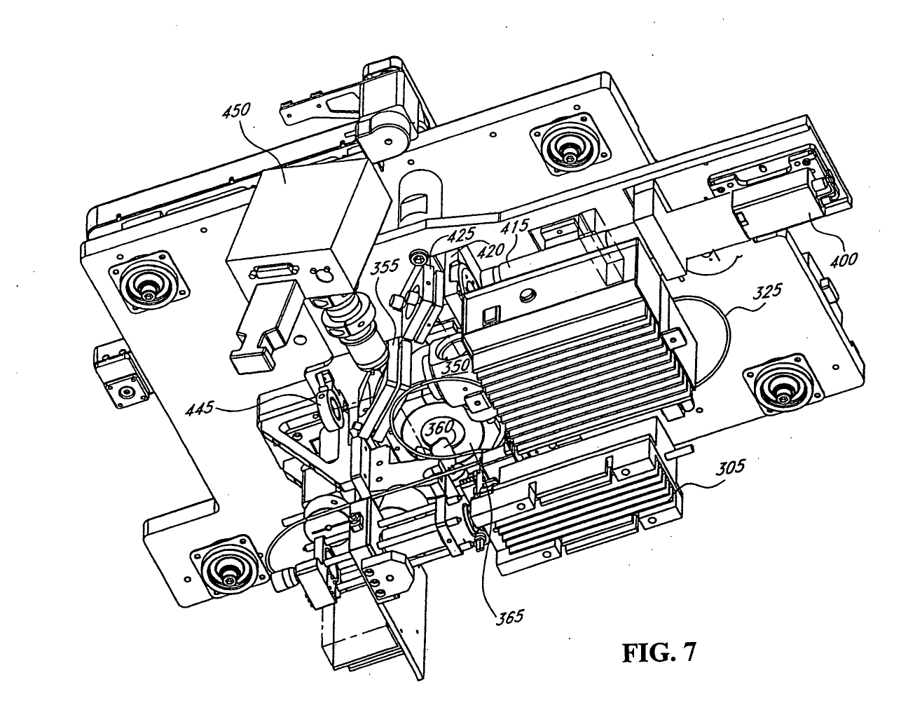

An interior view of the apparatus 10 is provided in FIG. 3. As illustrated, the apparatus 10 provides an upper tray 200 and lower tray 210 that hold the interior components of the apparatus. The upper tray 200 includes a pair of intake filters 215A,B that filter ambient air being drawn into the interior of the apparatus 10. Below the access door 35 is the optical subassembly (not shown). The optical subassembly is mounted to the upper tray 200 and is discussed in detail with regard to FIGS. 4-7.

On the lower tray 210 is a computer 225 which stores the software programs, commands and instructions that run the apparatus 10. In addition, the computer 225 provides control signals to the treatment apparatus through electrical signal connections for directing electromagnetic radiation from the laser energy source.

As used herein “computer” can be, without being limited to these, any microprocessor or processor controlled device, such as personal computers, workstations, servers, clients, mini computers, main-frame computers, laptop computers, a network of individual computers, mobile computers, palm-top computers, hand-held computers, set top boxes for a TV, interactive televisions, interactive kiosks, personal digital assistants, interactive wireless communications devices, mobile browsers, or a combination thereof. The computers can further possess input devices such as a keyboard, mouse, touchpad, joystick, pen-input-pad, and output devices such as a computer screen and a speaker. These computers may be uni-processor or multi-processor machines.

Additionally, these computers can include an addressable memory or storage medium or computer accessible medium, such as random access memory (RAM), an electronically erasable programmable read-only memory (EEPROM), programmable read-only memory (PROM), erasable programmable read-only memory (EPROM), hard disks, floppy disks, laser disk players, digital video devices, compact disks, video tapes, audio tapes, magnetic recording tracks, electronic networks, and other techniques to transmit or store electronic content such as, by way of example, programs and data. In some embodiments, the computers can be equipped with a network communication device such as a network interface card, a modem, or other network connection device suitable for connecting to a networked communication medium.

As illustrated, a series of power supplies 230A,B,C provide power to the various electrical components within the apparatus 10. In addition, an uninterruptable power supply 235 is incorporated to allow the apparatus to continue functioning through short external power interruptions.

FIG. 4 provides a layout of one embodiment of an optical subassembly design 300 within an embodiment of an apparatus 10. A laser 400 is present to irradiate the cells. As shown, the laser 400 outputs an energy beam of 523 nm that passes through a shutter 410. Although the exemplary laser outputs an energy beam having a 523 nm wavelength, other sources that generate energy at other wavelengths are also within the scope of the present invention.

Once the laser energy beam passes through the shutter 410, it enters a beam expander (Special Optics, Wharton, N.J.) 415 which adjusts the diameter of the energy beam to an appropriate size at the plane of the solid surface. Following the beam expander 415 is a half-wave plate 420 which controls the polarization of the beam. The laser energy beam is then reflected off a mirror 425 and enters the cube beamsplitter 350. The laser energy beam is reflected by 90° in the cube beamsplitter 350. From the cube beamsplitter 350, the laser beam reflects off the long wave pass mirror 355, is steered by the galvanometers 360, thereafter enters the scanning lens 365, and finally is focused within a defined volume.

A Nd:YLF frequency-doubled, solid-state laser (Spectra-Physics, Mountain View, Calif.) is used because of its stability, high repetition rate of firing, and long time of maintenance-free service. Other similar lasers, including the Nanolaser (JDS Uniphase, San Jose, Calif.) Nd:YAG first harmonic (1064 nm), Nd:YAG second harmonic (532 nm), and Nd:YAG third harmonic (355 nm) versions can also be used in this apparatus.

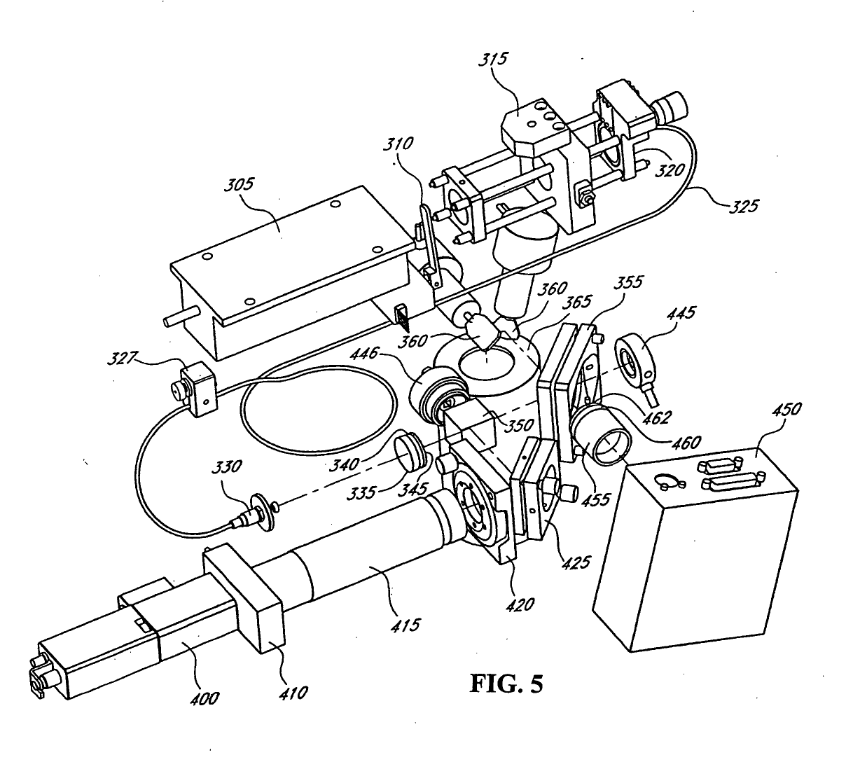

Referring now to FIG. 5, a perspective view of an embodiment of an optical subassembly is illustrated. As illustrated in the perspective drawing of FIG. 5, the laser 400 transmits energy through the shutter 410 and into the beam expander 415. Energy from the laser 400 passes through the beam expander 415 and passes through the half-wave plate 420 before hitting the fold mirror 425, entering the cube beamsplitter 350 where it is reflected 90° to the long wave pass mirror 355, from which it is reflected into the computer controlled galvanometer mirrors 360. After being steered by the galvanometer mirrors 360 through the scanning lens 365, the laser energy beam strikes the defined volume in order to induce permeabilization of any cells present within.

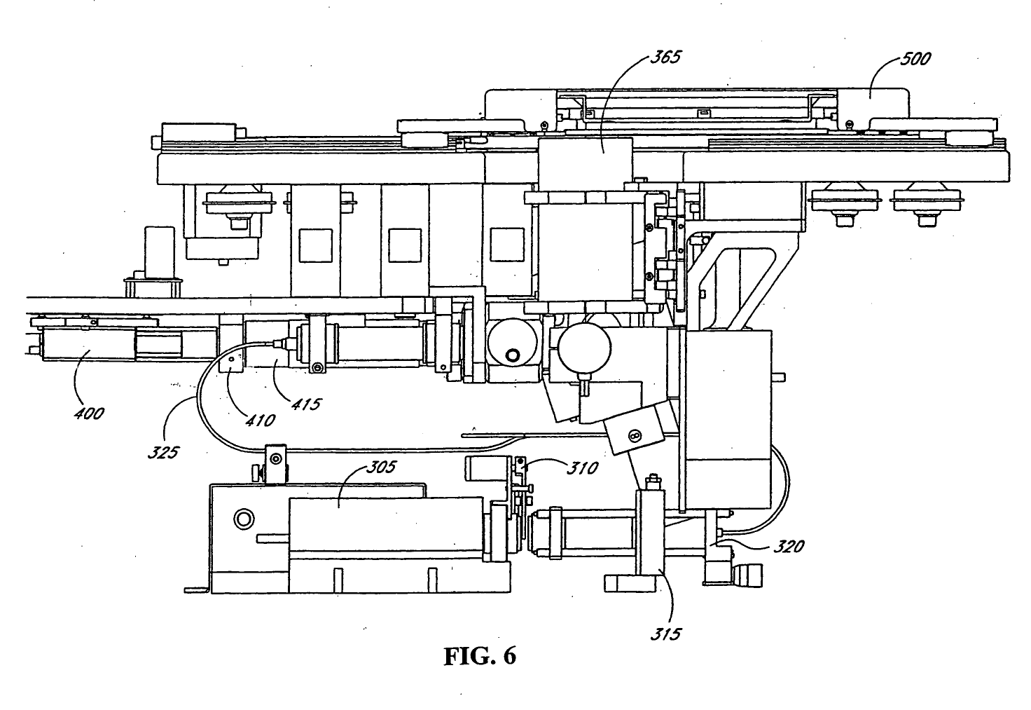

In order to accommodate a very large surface area of specimen to treat, the apparatus includes a movable stage that mechanically moves the specimen container with respect to the scanning lens. Thus, once a specific area of the solid surface has been treated, the movable stage brings another area of the solid surface within the scanning lens field-of-view. As illustrated in FIG. 6, a computer-controlled movable stage 500 holds a container (not shown) to be processed. The movable stage 500 is moved by computer-controlled servo motors along two axes so that the specimen container can be moved relative to the optical components of the instrument. The stage movement along a defined path is coordinated with other operations of the apparatus. In addition, specific coordinates can be saved and recalled to allow return of the movable stage to positions of interest. Encoders on the x and y movement provide closed-loop feedback control on stage position.

The flat-field (F-theta) scanning lens 365 is mounted below the movable stage. The lens 365 is mounted to a stepper motor that allows the lens 365 to be automatically raised and lowered (along the z-axis) for the purpose of focusing the system to the defined volume.

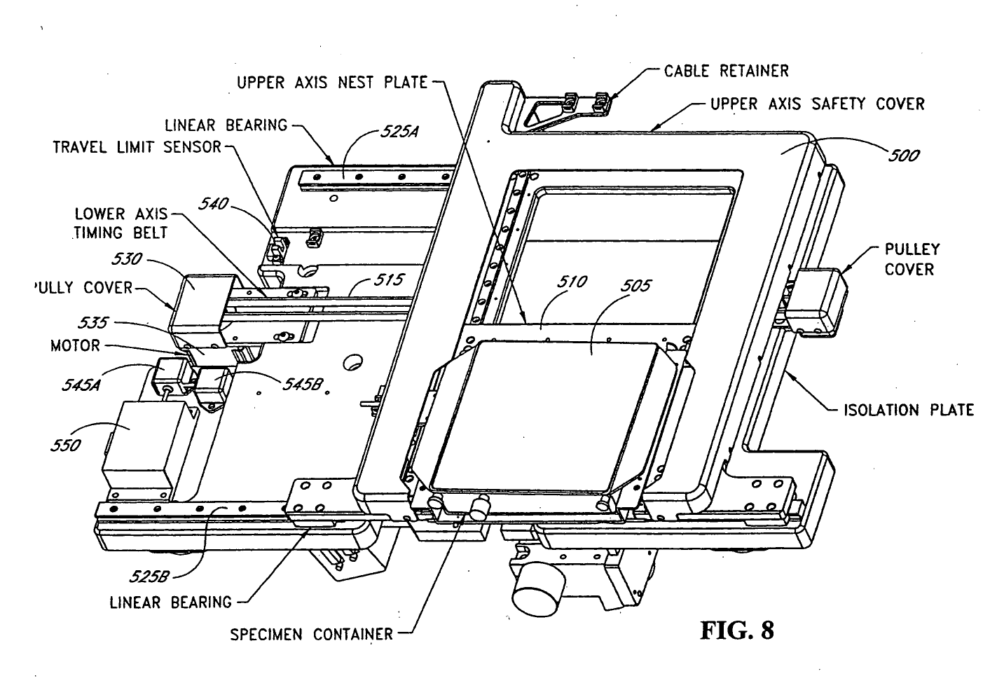

Referring now to FIG. 8, a top view of the movable stage 500 is illustrated. As shown, a container is mounted in the movable stage 500. The container 505 rests on an upper axis nest plate 510 that is designed to move in the forward/backward direction with respect to the movable stage 500. A stepper motor (not shown) is connected to the upper axis nest plate 510 and computer system so that commands from the computer cause forward/backward movement of the specimen container 505.

The movable stage 500 is also connected to a timing belt 515 that provides side-to-side movement of the movable stage 500 along a pair of bearing tracks 525A,B. The timing belt 515 attaches to a pulley (not shown) housed under a pulley cover 530. The pulley is connected to a stepper motor 535 that drives the timing belt 515 to result in side-to-side movement of the movable stage 500. The stepper motor 535 is electrically connected to the computer system so that commands within the computer system result in side-to-side movement of the movable stage 500. A travel limit sensor 540 connects to the computer system and causes an alert if the movable stage travels beyond a predetermined lateral distance.

A pair of accelerometers 545A,B is preferably incorporated on this platform to register any excessive bumps or vibrations that may interfere with the apparatus operation. In addition, a two-axis inclinometer 550 is preferably incorporated on the movable stage to ensure that the container is level, thereby reducing the possibility of gravity-induced motion in the container.

The chamber has a fan with ductwork to eliminate condensation on the container, and a thermocouple to determine whether the chamber is within an acceptable temperature range. Additional fans are provided to expel the heat generated by the electronic components, and appropriate filters are used on the air intakes 215A,B.

The computer system 225 controls the operation and synchronization of the various pieces of electronic hardware described above. The computer system can be any commercially available computer that can interface with the hardware. The computer can use any suitable operating system, including without limitation, for example, as Linux, Unix, Microsoft® Windows®, Apple® MacOS®, and IBM® OS/2®. One example of such a computer system is an Intel Pentium II-based computer running the Microsoft Windows® NT operating system. Another example of a computer system is one having a Pentium III or IV processor and one that runs a Windows® XP operating system. Software is used to communicate with the various devices, and control the operation in the manner that is described below.

Once a container is in place on the movable stage and the door is closed, the computer passes a signal to the stage to move into a home position. The fan is initialized to begin warming and defogging of the container. During this time, cells within the container are allowed to settle to the solid surface. In addition, during this time, the apparatus may run commands that ensure that the container is properly loaded, and is within the focal range of the system optics. For example, specific markings on the container can be located and focused on by the system to ensure that the scanning lens has been properly focused on the bottom of the container. After a suitable time, the computer turns off the fan to prevent excess vibrations during treatment, and processing begins.

The operator can direct operation of the apparatus via the keyboard and mouse, for example, by selecting which area of the container to expose to electromagnetic radiation. The computer then instructs the movable stage to be positioned over the scanning lens so that the first area of the container to be exposed is directly in the scanning lens field-of-view. The laser begins firing at a pre-determined rate, and each laser pulse is directed to a different defined volume by movement of the galvanometer mirrors. Due to the speed of the laser and galvanometers, thousands of pulses can be directed to thousands of defined volumes per second, thereby enabling high-throughput permeabilization of cell membranes. One brand of galvanometer is the Cambridge Technology, Inc. model number 6860 (Cambridge, Mass.). This galvanometer can reposition very accurately within a millisecond, making the processing of large areas possible within a reasonable amount of time. Error signals continuously generated by the galvanometer control boards are monitored by the computer to ensure that the mirrors are in position and stable before the laser is fired, in a closed-loop fashion.

It should be noted and understood that the system depicted in FIGS. 2-8 can be modified. For example, the methods and apparatuses do not require some of the optical components, such as the camera and illumination sources, although those can be used with the embodiments disclosed herein.

Other embodiments relate to systems with a memory, where the memory includes a set of instructions. The instructions when executed can cause a computer to perform an action. The action can include directing to a solid surface electromagnetic radiation sufficient to induce permeabilization of a membrane of a substantially stationary cell, without prior knowledge of the specific three-dimensional location of said cell, wherein the cell is coincident with the path of said electromagnetic radiation. The action can also include directing electromagnetic radiation to a location within a defined volume without regard to the characteristics of the location. The “characteristics of the location” can include knowledge of whether a cell is located at a particular location, for example based upon visualization of the location, color of the location, fluorescence of the location, light transmission, and the like, for example. This is in contrast to systems that direct radiation to a particular location based upon fluorescent labels, visual images, dyes, and the like. The amount of electromagnetic radiation can be sufficient to induce permeabilization of a membrane of a cell that is coincident with the electromagnetic radiation.

The memory can be any suitable memory, including any as described above. The set of instructions can be C++ code, any other code, initialization files, analogue circuits, and the like. The computer can be any computer, including those described above using any operating system, including the Windows XP® operating system.

The following examples illustrate the use of the described method and apparatus in different applications.

### EXAMPLES

### Example 1

### Loading of Cells with Nucleic Acids

Since the recent discovery of effective RNA interference (RNAi)-mediated gene silencing in mammalian cells (Elbashir, S. M., Harborth, J., Lendeckel, W., Yalcin, A., Weber, K., & Tuschl, T. 2001. Duplexes of 21-nucleotide RNAs mediate RNA interference in cultured mammalian cells. Nature, 411: 494-498; which is incorporated herein by reference in its entirety), there has been significant validation and interest in the approach from both academic and corporate researchers, for both discovery and therapeutic applications. RNAi has a number of advantages over older antisense technologies for gene silencing, which have led to many recent reports in a number of useful model systems. However, nucleic acids do not readily pass through intact living cell membranes, and most of the reports to date have described limitations with respect to using existing cell transfection methods for implementing RNAi. Although RNAi is a potentially powerful tool, there is no high-efficiency and high-viability transfection method available. Laser-mediated optoinjection has the potential to overcome many of the limitations associated with other techniques.

In this example, DNA that codes for the sense and antisense RNAi strands was purchased from Allele Biotechnology (San Diego, Calif.). This approach is based on RNAi cassettes where U6 RNA-based polymerase III promoter and modified terminator is used for high level and specific RNAi expression inside the cell. DNA template and upstream primers were provided with the kit. Downstream GFP specific primers, both sense and antisense, were used to generate sense and antisense small interfering RNA (siRNA) transcripts by PCR using conditions per the manufacturer's recommendations (LineSilence™ Kit, Allele Biotechnology, San Diego, Calif.). The following 54 bp DNA sequences represent the terminator, gene-specific downstream sequences, and template matching region.

(SEQ ID NO:1)

5′-caaaaactgtaaa AA GAACGGCATCAAGGTGAA C ggtgtttcg
tcctttccaca-3′

(SEQ ID NO:2)

5′-caaaaactgtaaa AA GTTCACCTTGATGCCGTT C ggtgtttcg
tcctttccaca-3′

The PCR products were then purified, annealed and used in the optoinjection studies to achieve gene silencing of a GFP reporter gene.

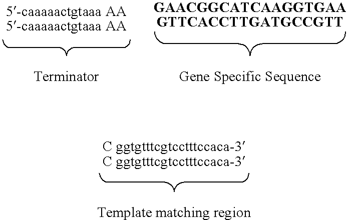

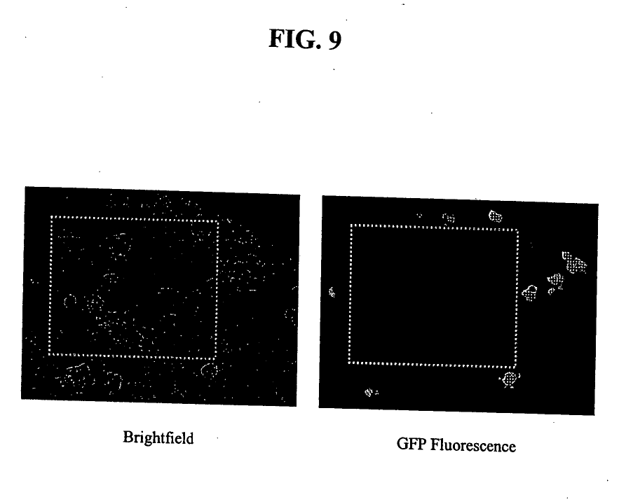

293T-GFP cells (293T obtained from ATCC, Manassas, Va. and transfected with phrGFP-1 (Stratagene, La Jolla, Calif.)) grown in RPMI 1640 with 10% FBS and 0.2 mg/ml G418 were trypsinized and plated into 384-well plates. The cells were incubated, allowed to attach for 24 hours, and then processed in situ by washing once with PBS, and then adding sense and antisense PCR oligos (10-25 ng) in 5 μl (microliters) Hypo-osmolar Buffer (Brinkman, Westbury, N.Y.). An area of the well was exposed to a predetermined grid pattern of laser shots that did not require locating the target cells prior to shooting. For reference only, the perimeter of this area (approximately 0.0001 square centimeters) is shown in the dotted box in FIG. 9. Because these cells grow attached to the solid surface, the effective distance was a few micrometers. A 523 nm wavelength pulsed laser beam of 10μ (micro) J/pulse and 10 nanosecond pulse width was focused down to 30μ (micro) m in diameter (yielding an energy density of 0.007 μJ/μm² per pulse and a peak power density of 7×10⁷ W/cm², considering the 50% transmission efficiency to the defined volume in the specimen), and pulses were fired and steered sequentially such that the distance between adjacent shots within the predetermined grid pattern was 20 μm in both x- and y-directions. The laser pulses were fired and steered at a rate of 300 per second, leading to irradiation of the entire defined volume in less than one-tenth of a second. This method resulted in irradiation of every cell within the defined volume without prior knowledge of the cells' locations. The image shown in FIG. 9 is merely for reference, and was not used to target the cells or area. Immediately following laser treatment, the buffer was removed and replaced with growth medium and the plates were directly placed in the incubator. After 48 hours, Propidium Iodide was used to confirm the viability of cells at >70%, and fluorescent imaging was used to confirm the silencing of the GFP gene in cells within the box. In the control area of the well outside the defined volume (not laser-irradiated, but exposed to the same reagents), most cells express GFP. Within the defined volume, all cells (approximately 30 in number) have markedly reduced GFP expression. The results show that cells within the defined volume were successfully permeabilized and loaded with DNA, leading to gene silencing of GFP.

### Example 2

### Loading of Cells with siRNA

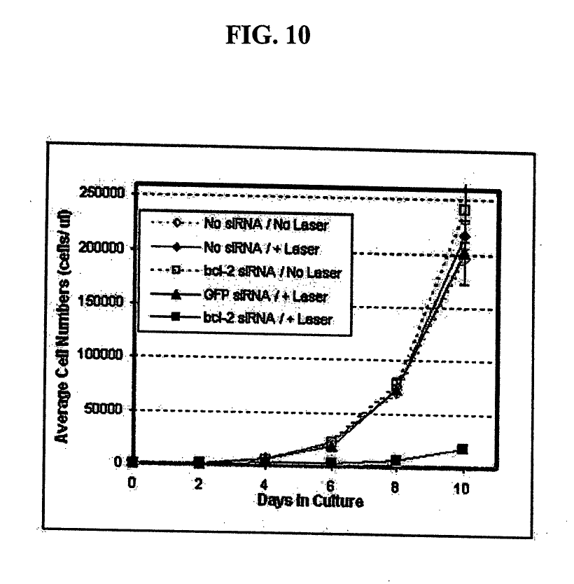

In this example, silencing of the bcl-2/IgH gene in SU-DHL-6 cells was achieved with optoinjection of siRNA leading to suppressed cell growth (FIG. 10), clearly demonstrating the delivery of a functional siRNA to affect cell function. Cells were grown in 384 well plates with RPMI 1640 and 10% FBS at 500 cells per well. siRNA encoding for bcl-2 was added at a concentration of 10 nM in PBS with 1% HSA. In this example, the defined volume comprised the entire area of the well (approximately 0.03 square centimeters), and an effective distance of approximately 10-20 micrometers because SU-DHL-6 cells do not grow attached to the solid surface. Cells were optoinjected using shots of 532 nm light in a 25 μm diameter beam, in a grid pattern with 25 micrometer spacing, at 10 μJ per pulse and 0.5 nanosecond pulse width (yielding an energy density of 0.01 μJ/μm² per pulse and a peak power density of 2×10⁹ W/cm², considering the 50% transmission efficiency to the defined volume in the specimen). In this example, pulses were fired and steered at a rate of 1,200 per second, such that the defined volume was irradiated in approximately 4 seconds. Cells were washed immediately after optoinjection, growth medium was added, and the cells were then incubated. After 24 hours, the cell viability was greater than 50%. Cells were incubated for a total of ten days with cell counts performed at days 2, 4, 6, 8, and
10. In addition to effective cell permeabilization, loading, and gene silencing, these data indicate normal cell growth following optoinjection without siRNA present, or with siRNA against an irrelevant target (i.e., GFP).

This method of cell transfection is very simple, rapid, and benign (>90% viability, no change in cell growth rate), and has been applied to a wide variety of reagents (e.g., plasmids, oligonucleotides, small organic molecules, ions, etc.).

### Example 3

### Loading of Cells with Zinc

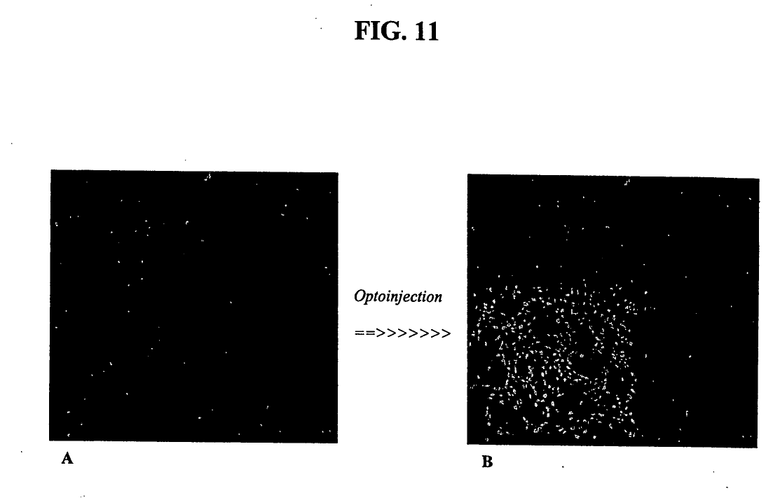

To demonstrate that ions from the extracellular medium could be loaded into cells, Zn²⁺, which has very low intracellular abundance, was selected for optoinjection. NIH-3T3 cells were first stained with a Zn²⁺-sensitive indicator (RhodZin-1; Molecular Probes, Inc. Eugene, Oreg.) using PBS with [Zn²⁺]ₒ=1 mM as the buffer. The perimeter of the defined area (approximately 0.001 square centimeters) is clearly visible in FIG. 11. Because these cells grow attached to the solid surface, the effective distance was a few micrometers. A 523 nm wavelength pulsed laser beam of 2 μJ/pulse and 10 nanosecond pulse width was focused down to 30 μm in diameter (yielding an energy density of 0.001 μJ/μm² per pulse and a peak power density of 1×10⁷ W/cm², considering the 50% transmission efficiency to the defined volume in the specimen), and pulses were fired and steered sequentially such that the distance between adjacent shots within the predetermined grid pattern was about 50 μm in both x- and y-directions. FIG. 11 shows a fluorescent image excited at 530 nm with emission detected at 590 nm. Panel A shows that cells at basal [Zn²⁺]_{I} about 0 have a very low fluorescence intensity. Panel B shows cells after optoinjection, wherein cells in the defined volume (i.e., the lower left corner) have increased RhodZin-1 fluorescence intensity due to increased [Zn²⁺]ᵢ. Cell viability was determined to be 90% under these conditions. This result demonstrates that laser irradiation of cells within the defined volume, in the presence of high [Zn²⁺]ₒ, caused increased [Zn²⁺]ᵢ. This experiment further indicates that influx of ions into the cytosol is from the extracellular medium rather than from intracellular stores.

Although aspects of the present invention have been described by particular embodiments exemplified herein, the present invention is not so limited.

### CITED REFERENCES

All of the references cited below and herein are incorporated herein by reference in their entireties.
Elbashir, S. M., Harborth, J., Lendeckel, W., Yalcin, A., Weber, K., & Tuschl, T. 2001. Duplexes of 21-nucleotide RNAs mediate RNA interference in cultured mammalian cells. Nature, 411: 494-498.
Guo, Y., Liang, H., & Berns, M. W. 1995. Laser-mediated gene transfer in rice. Physiol.Plant, 93: 19-24.
Han, M., Gao, X., Su, J. Z., & Nie, S. 2001. Quantum-dot-tagged microbeads for multiplexed optical coding of biomolecules. Nat.Biotech., 19: 631-635.
Koller, M. R., Hanania, E. G., Eisfeld, T. M., & Palsson, B. O., U.S. Patent Application Publication No. 20020076744, published on Jun. 20, 2002 entitled Optoinjection methods, for U.S. patent application Ser. No. 09/961,691 filed Sep. 21, 2001.
Krasieva, T. B., Chapman, C. F., LaMorte, V. J., Venugopalan, V., Berns, M. W., & Tromberg, B. J. 1998. Mechanisms of cell permeabilization by laser microirradiation. Proc.SPIE, 3260: 38-44.
Kurata, S., Tsukakoshi, M., Kasuya, T., & Ikawa, Y. 1986. The laser method for efficient introduction of foreign DNA into cultured cells. Exp.Cell Res., 162: 372-378.
Palsson, B. O., U.S. patent application Ser. No. 10/359,483, filed Feb. 4, 2003, entitled “Method and Apparatus for Selectively Targeting Specific Cells within a Cell Population.”
Palumbo G, Caruso M, Crescenzi E, Tecce M F, Roberti G, Colasanti A.
   1996 Targeted gene transfer in eucaryotic cells by dye-assisted laser optoporation. J Photochem Photobiol B. 36(1):41-6.
Shirahata, Y., Ohkohchi, N., Itagak, H., & Satomi, S. 2001. New technique for gene transfection using laser irradiation. J. Invest.Med., 49: 184-190.
Soughayer, J. S., Krasieva, T., Jacobson, S. C., Ramsey, J. M., Tromberg, B. J., & Allbritton, N. L. 2000. Characterization of cellular optoporation with distance. Anal.Chem., 72: 1342-1347.
Tao, W., Wilkinson, J., Stanbridge, E. J., & Berns, M. W. 1987. Direct gene transfer into human cultured cells facilitated by laser micropuncture of the cell membrane. PNAS, 84: 4180-4184.
Tirlapur, U. K. & Konig, K. 2002. Targeted transfection by femtosecond laser. Nature, 418: 290-291.
Tsukakoshi, M., Kurata, S., Nominya, Y., Ikawa, Y., & Kasuya, T. 1984. A novel method of DNA transfection by laser microbeam cell surgery. Appl.Phys., 35: 135-140.

What is claimed is:

1. A method of transiently permeabilizing one or more cells, comprising:
a) maintaining said one or more cells in a substantially stationary position within an effective distance from a solid surface; and
b) directing to said solid surface electromagnetic radiation sufficient to induce transient permeabilization of a membrane of said one or more cells, without prior knowledge of the specific three-dimensional location of said one or more cells, wherein said one or more cells is coincident with the path of said electromagnetic radiation.

2. The method of claim 1, wherein said electromagnetic radiation has an energy density at said solid surface selected from the group consisting of at most about 0.001, 0.002, 0.003, 0.006, 0.01, 0.02, 0.03, 0.06, 0.1, 0.2, 0.3, 0.6, 1, 2, 3, 4, 5 and 6 μJ/μm².

3. (canceled)

4. (canceled)

5. The method of claim 1, wherein said effective distance is between about 1 μm to about 20 μm.

6. (canceled)

7. The method of claim 1, wherein said one or more cells are exposed to said electromagnetic radiation for a period of time of about 100 picoseconds to about 10 seconds.

8. The method of claim 1, wherein said directing comprises delivering a pulse of radiation to said solid surface.

9. (canceled)

10. (canceled)

11. The method of claim 1, further comprising inducing transient permeabilization in a membrane of said one or more cells at a rate of between about 300 to about 10,000,000 cells per second.

12. (canceled)

13. The method of claim 1, wherein the probability of viability of said one or more cells after said transient permeabilizing is maintained at a value of at least 50% to at least 90%.

14. The method of claim 1, further comprising contacting said one or more cells with a non-isotonic aqueous medium.

15. (canceled)

16. (canceled)

17. The method of claim 1, further wherein said one or more cells contacts an aqueous medium such that a substance within said aqueous medium can enter said one or more cells through a transiently permeabilized membrane.

18. The method of claim 17, wherein said substance is selected from the group consisting of an ion, an organic molecule, an inorganic molecule, a colloidal particle, a polysaccharide, a peptide, a protein, a nucleic acid, and a modified nucleic acid.

19. (canceled)

20. (canceled)

21. (canceled)

22. The method of claim 1, wherein said electromagnetic radiation is directed to an area of said solid surface at a rate of about 0.0003 to about 10 square centimeters per second.

23. (canceled)

24. (canceled)

25. (canceled)

26. The method of claim 1, wherein said directing comprises delivering two or more pulses of electromagnetic radiation to said solid surface according to a pulse target pattern.

27. The method of claim 26, wherein an individual pulse of said pulses of electromagnetic radiation has a duration selected from the group consisting of at most on the order of 1000 seconds, 100 seconds, 10 seconds, 1 second, 100 milliseconds, 10 milliseconds, 1 millisecond, 100 microseconds, 10 microseconds, 1 microsecond, 100 nanoseconds, 10 nanoseconds, 1 nanosecond, 100 picoseconds, 10 picoseconds, 1 picosecond, 100 femtoseconds, 10 femtoseconds, 1 femtosecond, 100 attoseconds, 10 attoseconds, and 1 attosecond.

28. (canceled)

29. The method of claim 26, wherein at least two pulses of electromagnetic radiation are directed to a single pulse target within said pulse target pattern.

30. (canceled)

31. The method of claim 1, wherein said electromagnetic radiation is directed to a defined area on said solid surface, and said defined area has an area of about 0.0001 to about 10 square centimeters.

32. (canceled)

33. (canceled)

34. The method of claim 1, wherein said path of said electromagnetic radiation has a width of about 10 micrometers to about 1000 micrometers.

35. An apparatus for transiently permeabilizing a cell, comprising:
a) an energy source that emits electromagnetic radiation sufficient to induce transient permeabilization of a membrane of a cell, wherein said cell is substantially stationary and contained within a defined volume, wherein the specific coordinates of said cell within said defined volume are unknown, and wherein said defined volume is partly bounded by a solid surface;
b) a directing device configured to direct said electromagnetic radiation to substantially the entirety of said defined volume, wherein said cell is coincident with the path of said electromagnetic radiation, and wherein said electromagnetic radiation within said defined volume has an energy density at said solid surface of at most about 6 μJ/μm²; and
c) said solid surface.

36. (canceled)

37. The apparatus of claim 35, wherein said electromagnetic radiation within said defined volume has an energy density at said solid surface of about 0.001 to about 0.3 μJ/μm².

38. The apparatus of claim 35, wherein said directing device directs pulses of electromagnetic radiation to said defined volume according to a pulse target pattern.

39. (canceled)

40. The apparatus of claim 37, wherein an individual pulse of said pulses of electromagnetic radiation has a duration of about 10 seconds to about 100 picoseconds.

41. (canceled)

42. (canceled)

43. The apparatus of claim 35, wherein said path of said electromagnetic radiation has a width of about 10 micrometers to about 1000 micrometers.

44. A system with a memory comprising a set of instructions, such that when executed the computer performs the action comprising directing to a solid surface electromagnetic radiation sufficient to induce transient permeabilization of a membrane of a substantially stationary cell, without prior knowledge of the specific three-dimensional location of said cell, wherein said cell is coincident with the path of said electromagnetic radiation.


SEQUENCE LISTING (2 sequences)

SEQ ID NO: 1 - DNA, 54 bp, Artificial Sequence
  PCR products for use in RNA interference specific for GFP gene
caaaaactgtaaaaagaacggcatcaaggtgaacggtgtttcgtcctttccaca

SEQ ID NO: 2 - DNA, 54 bp, Artificial Sequence
  PCR products for use in RNA interference specific for GFP gene
caaaaactgtaaaaagttcaccttgatgccgttcggtgtttcgtcctttccaca